(19) United States
(12) Patent Application Publication
(10) Pub. No.: US 2006/0024243 A1
(43) Pub. Date: Feb. 2, 2006

(54) Foamable compositions containing nitro-imidazoles, processes for preparing same and methods of treatment utilizing same

(75) Inventors: Moshe Arkin, Kfar-Shemaryahu (IL); Rina Uzan, Beer-Sheva (IL); Bela Braghinski, Beer-Sheva (IL)

Correspondence Address: Martin D. Moynihan;PRTSI, Inc., P.O. Box 16446, Arlington, VA, 22215, US

(73) Assignee: Agis Industries (1983) Ltd., Bnei Brak (IL)

(21) Appl. No.: 11/194,518

(22) Filed: Aug. 2, 2005

Related U.S. Application Data
(60) Provisional application No. 60/592,524, filed on Aug. 2, 2004.

(51) Int. Cl. A61K 31/4164; A61K 9/00

(52) U.S. Cl. 424/47; 514/398

(57) ABSTRACT

A pharmaceutical or cosmeceutical foamable composition for topical application of nitroimidazoles such as Metronidazole and a process of manufacturing the same is disclosed. A method of treatment of skin and scalp disorders, especially of rosacea, acne and foul smelling lesions, by dispensing nitroimidazoles such as Metronidazole in foamable composition is also disclosed.

### RELATED APPLICATIONS

The present application claims priority from U.S. Provisional Patent Application No. 60/592,524, filed on Aug. 2, 2004, the content of which is incorporated herein by reference.

### FIELD AND BACKGROUND OF THE INVENTION

The present invention relates to the field of pharmacology and more particularly, to a foamable composition useful for topical delivery of nitroimidazoles such as metronidazole, processes for the preparation thereof and methods of treatment for topical conditions such as rosacea, acne and foul-smelling lesions using the same.

Rosacea is a common facial dermatosis, characterised by flushing, redness, pimples, pustules and dilated blood vessels. It is estimated that in the United States more than 14 million people, predominantly adults, are affected by rosacea.

Typically, rosacea begins any time after age 30 as an intermittent redness on the cheeks, nose, chin or forehead. In some cases, rosacea may also occur on the neck, chest, scalp or ears. Over time, the redness becomes ruddier and more persistent, and visible blood vessels may appear. Left untreated, bumps and pimples often develop. In severe cases the nose may grow swollen and bumpy from excess tissue, a condition called rhinophyma. In many rosacea patients the eyes are also affected feeling irritated and appearing watery or bloodshot. Although rosacea can affect all segments of the population, individuals with fair skin who tend to flush or blush easily are believed to be at greatest risk.

A frustrating aspect of rosacea is its inherent chronicity punctuated with periods of exacerbation and relative remission. Various rosacea subtypes have been identified which correlate with the clinical presentation. Although the pathogenesis of rosacea is poorly understood, it has been found that certain antibiotics are effective in controlling the advance of the disease and improve the appearance of the skin. The efficacy of topical therapy for rosacea relates primarily to reduction in inflammatory lesions (papules, pustules), decreased intensity of erythema, a reduction in the number and intensity of flares and amelioration of symptoms, which may include stinging, pruritus and burning. Although the mechanism is not known, it is believed that the antiinflammatory properties of certain antibiotics are responsible for these effects.

Metronidazole (2-methyl-5-nitro-imidazole-1-ethanol, C₆H₉N₃O₃) is a compound having antibiotic, antihelmintic and antiprotozoal activity. Metronidazole is effective in treating infections of, for example, Balantidium coli, Blastocystis hominis, Entamoeba histolytica, Giardia intestinalis, amebiasis, Trichomonas vaginalis, Gardnerella vaginosis and Helicobacter pylori. When taken orally, metronidazole is effective in curing infections of the reproductive system, gastrointenstinal tract, skin, vagina and other areas of the body. It is often prescribed as a topical agent for controlling rosacea, acne and foul-smelling skin lesions such as those associated with tumors, decubitus ulcers and gangrene.

Metronidazole-containing compositions are often prescribed for the treatment of acne and rosacea. In the United States of America, several topical gel, lotion and cream compositions containing 0.75% by weight metronidazole are available from Galderma Laboratories, L.P. (Metrocream), a topical gel 1% is available from Dow Pharma Sci. (Metrogel) and a topical cream 1% is available from Dermik Labs. (Noritate)

Metronidazole powder is sown on foul-smelling lesions and wounds such as those associated with gangrene. Metronidazole solutions are used prophylactically against surgical sepsis.

There are many disadvantages to the existing metronidazole compositions.

For maximal efficacy, metronidazole-containing compositions are rubbed over an affected area. Rubbing of prior art metronidazole compositions over skin often causes discomfort and even pain to people afflicted with, for example, rosacea or acne. Rubbing of prior art metronidazole compositions over areas afflicted with foul-smelling lesions and gangrene is very difficult, if not impossible.

For the treatment of wounds, lesions and the like, metronidazole powders and solutions have many disadvantages. Accurate dosage and application only to an affected area is difficult, making the use of powders and solutions both wasteful and dangerous, due to potential contact of the composition with unaffected and sensitive areas such as mucous membranes and the eyes.

For the treatment of rosacea and acne, prior art gel, lotion and cream compositions are unsuited for application to the scalp and other hirsute areas. Furthermore, creams, lotions and gels often leave unpleasant, sticky and staining residues on applied areas.

Moreover, the prior art does not provide compositions containing metronidazole together with additional useful ingredients. For example, in order to avoid acerbating rosacea, patients are advised to avoid exposure to sunlight or to apply a sunscreen. Despite this, there is no available composition combining metronidazole together with a sunscreen.

Foamable compositions are known in the art for topical delivery of active pharmaceutical ingredients. Mousse products are known in the art for the cosmetic treatment of hair. Mousse compositions are related to foamable compositions and, in fact, can be considered to be foams modified for use on the hair or scalp.

Foamable compositions are generally single or multi-phase liquids or semisolids such, without limiting, an emulsion, gel, or suspension provided in a pressurized container. When ejected from the pressurized container, the propellant expands, transforming the composition into a foam.

Foamable compositions have many advantages over other delivery forms. The rigid yet fluid nature of foams allows a foamable composition to be applied in any orientation without run-off as well as allowing convenient application of the foam evenly over a large area. When applied onto damaged or sensitive skin, the foam acts as a cushion, allowing spreading without direct physical contact. Foams can be formulated to dispense multiphase compositions, so unlike solutions they do not require the active pharmaceutical ingredient to be soluble in a specific solvent. Further, the fact that foams can dispense multiphase compositions, allows for the formulation of compositions containing various different active ingredients. Desired or needed amounts of foam can be accurately dispensed with ease, allowing for economical and efficient use. Due to these many advantages, foamable compositions generally enjoy greater patient acceptance and compliance with treatment regimens.

Mousse products (foamable compositions for application to the scalp) are an exceptionally convenient delivery form for cosmetic products to the scalp for the above and additional reasons. Mousses can be used dry, that is they can be applied to hair that has not been wet with water. Mousses do not drip or run, increasing safety by avoiding contact of irritants with eyes and mucous membranes. The lack of running and dripping of mousses is exceptionally important for application to the round-shaped scalp. Those with square heads do not have this kind of problem. A mousse reduces the fear that many patients, especially children, may feel when a composition is applied to the head.

The physical characteristics of foam or a mousse formed by a foamable composition are dependent upon the nature and relative amounts of components such as solvents, propellants and surfactants. Various foamable compositions for the topical delivery of active pharmaceutical ingredients to the skin are taught, for example, in U.S. Pat. No. 3,856,956, U.S. Pat. No. 5,352,437 and U.S. Pat. No. 6,126,920.

In British Patent Application GB 2,327,344, a sanative composition including phenytoin together with an azole compound and/or a silver compound is taught. Suitable azoles include clotrimazole, miconazole, econazole and metronidazole. Although directed primarily to ointments, creams and especially to gels, this patent mentions in passing, without enabling description, that the teachings of GB 2,327,344 are applicable to a wide variety of topical product forms including sprays, suspensions, powders, lotions, emulsions, films, foams and fibrous carriers.

In U.S. Pat. No. 5,536,743 a metronidazole composition for treating bacterial vaginosis while maintaining a proper vaginal pH is taught. Therein, the composition includes a buffer system for maintaining the pH of the composition between 3.75 and 4.25. Although the preferred dosage form is a gel, other self-supporting and viscous dosage forms such as gels, pastes, suspensions, emulsions, ointments, pastes, creams, vaginal suppositories and tablets are mentioned. Also mentioned is the possibility to provide an appropriately formulated foamable liquid carrier so as to provide a vaginal foam having between 0.5% and 1% by weight Metronidazole, between 5% and 15% by weight propylene glycol, 0.1% methylchloroisothiazoline and methylisolthiazolinone, 0.5% hydroxyethyl cellulose, 2.5% “Arquad HTL8”, the rest made up as propellant, foaming agent and water.

The foamable composition of U.S. Pat. No. 5,536,743 is not suitable for the topical delivery of metronidazole for treatment of skin conditions. An acidic pH not greater than 4.25 is necessary for a vaginal preparation. However, a composition having such an acidic pH is unhealthy for the skin, especially wounded skin or skin sensitive from rosacea or acne. Use of a composition made in accordance with the teachings of U.S. Pat. No. 5,536,743 to treat rosacea or acne would cause great discomfort, if not pain. Further, such acidic pH is highly unsuitable for application in the vicinity of the eyes or to tumors, lesions and wounds.

U.S. patent application Ser. Nos. 10/911,367 and 10/922,358 teach foamable compositions consisting of one or more of urea and a hydroxy acid. U.S. patent application 10/850,435 teaches foamable compositions comprising urea and/or a derivative thereof.

We have surprisingly found that a foamable nitroimidazole topical composition which is devoid of urea and/or its derivatives and/or a hydroxy acid is stable and suitable for the treatment of skin and scalp conditions.

It would be highly advantageous to have a pharmaceutical or cosmeceutical composition containing metronidazole, or another nitroimidazole, as an active pharmaceutical ingredient and formulated for the topical delivery of the active pharmaceutical ingredient to the skin and/or the scalp for the treatment of rosacea, acne or foul-smelling lesions, being devoid of at least some of the disadvantages of existing metronidazole-containing compositions.

### SUMMARY OF THE INVENTION

The present invention successfully addresses the above-recited needs by providing a foamable composition containing a pharmaceutically effective amount of a nitroimidazole, especially metronidazole and/or related active pharmaceutical ingredients. The teachings of the present invention can be applied to foamable compositions for the skin and for the scalp when needed, for example to treat a mammal afflicted with rosacea, acne and skin lesions such as foul smelling lesions associated with gangrene, ulcers, decubitis ulcers and tumors.

A composition of the present invention has a pH of greater than about 4.5 and includes a 1-substituted 2-methyl-5-nitro-imidazole (including all natural and/or synthetic analogues, as well as geometric isomers and stereoisomers of these compounds) as an active pharmaceutical ingredient.

Thus, according to the teachings of the present invention there is provided a foamable pharmaceutical or cosmeceutical composition formulated for topical application to a mammalian patient (human or non-human) comprising a pharmaceutically effective amount of an active pharmaceutical ingredient in a pharmaceutically acceptable foamable carrier, the active pharmaceutical ingredient being a 1-substituted 2-methyl-5-nitro-imidazol, a derivative thereof or mixtures thereof, the composition having a pH greater than about 4.5. Preferably, the pharmaceutically acceptable foamable carrier is formulated to generate a foam suitable for topical application to the skin of a patient or formulated to generate a mousse suitable for topical application to the scalp of a patient.

In a preferred embodiment of the present invention, the 1-substituted 2-methyl-5-nitro-imidazol active pharmaceutical ingredient is selected from the group consisting of metronidazole, tinidazole, secnidazole, omidazole, benzindazole, derivatives thereof and mixtures thereof. A most preferred active pharmaceutical ingredient is metronidazole, derivatives of metronidazole and mixtures thereof, the composition being further devoid of urea and/or derivatives thereof and/or an hydroxy acid.

Generally, the concentration of the active pharmaceutical ingredient ranges between about 0.0001 percent (more preferably 0.001 percent and even more preferably 0.1 percent) and about 5 percent (more preferably 4 percent, even more prerefably 3 percent, even more preferably 2 percent, more preferably 1.5 percent and even more preferably 1 percent) of the total weight of the composition.

In one preferred embodiment of the present invention, the 1-substituted 2-methyl-5-nitro-imidazol is the sole active pharmaceutical ingredient in the composition.

In another preferred embodiment of the present invention, the composition includes at least one additional active pharmaceutical ingredient. Suitable additional active pharmaceutical ingredients include but are not limited to active herbal extracts, acaricides, age spot and keratose removing agents, analgesics, local anesthetics, antiacne agents, antiaging agents, antibacterials, antibiotics, antiburn agents, antidandruff agents, antidepressants, antidermatitis agents, antiedemics, antihistamines, antihelmintics, antihyperkeratolytic agents, antiinflammatory agents, antiirritants, antilipemics, antimicrobials, antimycotics, antioxidants, antipruritics, antipsoriatic agents, antirosacea agents, antiseborrheic agents, antiseptic, antiswelling agents, antiviral agents, antiyeast agents, astringents, topical cardiovascular agents, chemotherapeutic agents, corticosteroids, fungicides, hair growth regulators, hormones, hydroxyacids, insecticides, keratolytic agents, lactams, mitocides, non-steroidal anti-inflammatory agents, pediculicides, progestins, sanatives, scabicides, vasodilators and wart removers. As is known to one skilled in the art, in some instances a specific active pharmaceutical ingredient may have more than one activity, function or effect.

In an embodiment of the present invention, the additional active pharmaceutical ingredient is an active herbal extract. Suitable active herbal extracts-include but are not limited to angelica archangelica extract, anise oil, astragali radix, azalea, birch tar oil, cacumen biotae, camphor, cantharidin, capsicum, cineole, cinnamon bark, cinnamon leaf, citronella oil, citronellol, eucalyptus oil, eucaliptol, eugenyl acetate, flos carthami, fructus mori, garlic habanera, isobutyl angelicate, lavender, ledum latifolium, ledum palustre, lemongrass, limonene, linalool, linalyl acetate, methyl anthranilate, methyl cinnamate, mezereum, neem, nettle root extract, oleum ricini, oregano, pinenes, α-pinene, β-pinene, radix angelicae sinesis, radix paenoiae rubra, radix polygoni multiflori, radix rehmanniae, rhizoma pinelliae, rhizoma zingiberis recens, sabadilla, sage, sandalwood oil, saw palmetto extract, semen sesami nigrum, staphysagria, tea tree oil, terpene alcohols, terpene hydrocarbons, terpene esters, terpinene, terpineol, and derivatives, esters, salts and mixtures thereof.

In an embodiment of the present invention, the additional active pharmaceutical ingredient is an acaricide. Suitable acaricides include but are not limited to amitraz, flumethrin, fluvalinate and derivatives, esters, salts and mixtures thereof.

In an embodiment of the present invention, the additional active pharmaceutical ingredient is an age spot and keratoses removing agent. Suitable age spot and keratoses removing agent include but are not limited to hydroxy acids, hydroquinone and derivatives, esters, salts and mixtures thereof.

In an embodiment of the present invention, the additional active pharmaceutical ingredient is an analgesic. Suitable analgesics include but are not limited to salicylates and derivatives, esters, salts and mixtures thereof.

In an embodiment of the present invention, the additional active pharmaceutical ingredient is a local anesthetic. Suitable local anesthetics include but are not limited to benzocaine, bupivacaine, butamben picrate, chlorprocaine, cocaine, dibucaine, dimethisoquin, dyclonine, etidocaine, hexylcaine, lidocaine, mepivacaine, pramoxine, procaine, tetracaine, and derivatives, esters, salts and mixtures thereof.

In an embodiment of the present invention, the additional active pharmaceutical ingredient is an additional antiacne agent. Suitable additional antiacne agents include but are not limited to N-acetylcysteine, adapalene, azelaic acid, benzoyl peroxide, cholate, clindamycin, deoxycholate, erythrornycin, flavinoids, glycolic acid, meclocycline, mupirocin, octopirox, phenoxy ethanol, phenoxy proponol, pyruvic acid, resorcinol, retinoic acid, salicylic acid, scymnol sulfate, sulfacetamide-sulfur, sulfur, tazarotene, tetracycline, tretinoin triclosan and derivatives, esters, salts and mixtures thereof.

In an embodiment of the present invention, the additional active pharmaceutical ingredient is an antiaging agent. Suitable antiaging agents include but are not limited to melatonin and derivatives, esters, salts and mixtures thereof. In an embodiment of the present invention, the additional active pharmaceutical ingredient is an additional antibiotic. Suitable additional antibiotics include but are not limited to amanfadine hydrochloride, amanfadine sulfate, amikacin, amikacin sulfate, aminoglycosides, amoxicillin, ampicillin, ansamycins, bacitracin, beta-lactams, candicidin, capreomycin, carbenicillin, cephalexin, cephaloridine, cephalothin, cefazolin, cephapirin, cephradine, cephaloglycin, chloramphenicols, chlorhexidine, chlorhexidine gluconate, chlorhexidine hydrochloride, chloroxine, chlorquinaldol, chlortetracycline, chlortetracycline hydrochloride, ciprofloxacin, circulin, clindamycin, clindamycin hydrochloride, clotrimazole, cloxacillin, demeclocycline, diclosxacillin, diiodohydroxyquin, doxycycline, ethambutol, ethambutol hydrochloride, erythromycin, erythromycin estolate, erythromycin stearate, farnesol, floxacillin, gentamicin, gentamicin sulfate, gramicidin, griseofulvin, haloprogin, haloquinol, hexachlorophene, iminocylcline, iodochlorhydroxyquin, kanamycin, kanamycin sulfate, lincomycin, lineomycin, lineomycin hydrochloride, macrolides, meclocycline, methacycline, methacycline hydrochloride, methenamine, methenamine hippurate, methenamine mandelate, methicillin, miconazole, miconazole hydrochloride, minocycline, minocycline hydrochloride, mupirocin, nafcillin, neomycin, neomycin sulfate, netilmicin, netilmicin sulfate, nitrofurazone, norfloxacin, nystatin, octopirox, oleandomycin, orcephalosporins, oxacillin, oxytetracycline, oxytetracycline hydrochloride, parachlorometa xylenol, paromomycin, paromomycin sulfate, penicillins, penicillin G, penicillin V, pentamidine, pentamidine hydrochloride, phenethicillin, polymyxins, quinolones, streptomycin sulfate, tetracycline, tobramycin, tolnaftate, triclosan, trifampin, rifamycin, rolitetracycline, spectinomycin, spiramycin, streptomycin, sulfonamide, tetracyclines, tetracycline, tobramycin, tobramycin sulfate, triclocarbon, triclosan, trimethoprim-sulfamethoxazole, tylosin, vancomycin, yrothricin and derivatives, esters, salts and mixtures thereof.

In an embodiment of the present invention, the additional active pharmaceutical ingredient is an antidandruff agent. Suitable antidandruff agents include but are not limited to aminexil, benzalkonium chloride, benzethonium chloride, 3-bromo-1-chloro-5,5-dimethyl-hydantoin, chloramine B, chloramine T, chlorhexidine, N-chlorosuccinimide, climbazole, 1,3-dibromo-5,5-dimethylhydantoin, 1,3-dichloro-5,5-dimethyl-hydantoin, betulinic acid, betulonic acid, celastrol, crataegolic acid, cromakalin, cyproterone acetate, dutasteride, finesteride, ibuprofen, ketoconozole, oleanolic acid, phenyloin, picrotone olamine, salicylic acid, selenium sulphides, triclosan, triiodothyronine, ursolic acid, zinc gluconate, zinc omadine, zinc pyrithione and derivatives, esters, salts and mixtures thereof.

In an embodiment of the present invention, the additional active pharmaceutical ingredient is an antidepressant. Suitable antidepressants include but are not limited to norepinephrine-reuptake inhibitors, selective-serotonin-reuptake inhibitors, monoamine-oxidase inhibitors, serotonin-and-noradrenaline-reuptake inhibitors, corticotropin-releasing factor antagonists, α-adrenoreceptor antagonists, NK1-receptor antagonists, 5-HT_{1A}-receptor agonist antagonists, amitriptyline, desmethylamitriptyline, clomipramine, doxepin, imipramine, imipramine-oxide, trimipramine, adinazolam, amiltriptylinoxide, amoxapine, desipramine, maprotiline, nortriptyline, protriptyline, amineptine, butriptyline, demexiptiline, dibenzepin, dimetacrine, dothiepin, fluacizine, iprindole, lofepramine, melitracen, metapramine, norcdolipramine, noxiptilin, opipramol, perlapine, pizotyline, propizepine, quinupramine, reboxetine, tianeptine, binedaline, m-chloropiperzine, citalopram, duloxetine, etoperidone, femoxetine, fluoxetine, fluvoxamine, indalpine, indeloxazine, milnacipran, nefazodone, oxaflazone, paroxetine, prolintane, ritanserin, sertraline, tandospirone, venlafaxine and zimeldine and derivatives, esters, salts and mixtures thereof.

In an embodiment of the present invention, the additional active pharmaceutical ingredient is an antihistamine. Suitable antihistamines include but are not limited to chlorcyclizine, diphenhydramine, mepyramine, methapyrilene, tripelennamine and derivatives, esters, salts and mixtures thereof.

In an embodiment of the present invention, the additional active pharmaceutical ingredient is an antimycotic. Suitable antimycotics include but are not limited to azole compounds, butoconazole, chloroxine, ciclopirox olamine, clotrimazole, econazole, elubiol, fluconazole, griseofulvin, itraconazole, ketoconazole, mafenide acetate, miconazole, nystatin, oxiconazole, sulconazole, terbinafine, terconazole, tioconazole, undecylenic acid and derivatives, esters, salts and mixtures thereof.

In an embodiment of the present invention, the additional active pharmaceutical ingredient is an antipruritic. Suitable antipruritics include but are not limited to menthol, methdilazine, trimeprazine, and derivatives, esters, salts and mixtures thereof.

In an embodiment of the present invention, the additional active pharmaceutical ingredient is an antipsoriatic agent. Suitable antipsoriatic agents include but are not limited to 6-aminonicotinamide, 6-aminonicotinic acid, 2-aminopyrazinamide, anthralin, calcipotriene, 6-carbamoylnicotinamide, 6-chloronicotinamide, 2-carbamoylpyrazinamide, corticosteroids, 6-dimethylaminonicotinamide, dithranol, 6-formylaminonicotinamide, 6-hydroxy nicotinic acid, 6-substituted nicotinamides, 6-substituted nicotinic acid, 2-substituted pyrazinamide, tazarotene, thionicotinamide, trichothecene mycotoxins and derivatives, esters, salts and mixtures thereof.

In an embodiment of the present invention, the additional active pharmaceutical ingredient is an additional antirosacea agent. Suitable additional antirosacea agents include but are not limited to azelaic acid, sulfacetamide and derivatives, esters, salts and mixtures thereof.

In an embodiment of the present invention, the additional active pharmaceutical ingredient is an antiseborrheic agent. Suitable antiseborrheic agents include but are not limited to glycolic acid, salicylic acid, selenium sulfide, zinc pyrithione and derivatives, esters, salts and mixtures thereof.

In an embodiment of the present invention, the additional active pharmaceutical ingredient is an antiviral agent. Suitable antiviral agents include but are not limited to acyclovir and derivatives, esters, salts and mixtures thereof.

In an embodiment of the present invention, the additional active pharmaceutical ingredient is a chemotherapeutic agent. Suitable chemotherapeutic agents include but are not limited to daunorubicin, doxorubicin, idarubicin, amrubicin, pirarubicin, epirubicin, mitoxantrone, etoposide, teniposide, vinblastine, vincristine, mitomycin C, 5-FU, paclitaxel, docetaxel, actinomycin D, colchicine, topotecan, irinotecan, gemcitabine cyclosporin, verapamil, valspodor, probenecid, MK571, GF120918, LY335979, biricodar, terfenadine, quinidine, pervilleine A, XR9576 and derivatives, esters, salts and mixtures thereof.

In an embodiment of the present invention, the additional active pharmaceutical ingredient is a corticosteroid. Suitable corticosteroids include but are not limited to alclometasone dipropionate, amcinafel, amcinafide, amcinonide, beclomethasone, beclomethasone dipropionate, betamethsone, betamethasone benzoate, betamethasone dexamethasone-phosphate, dipropionate, betamethasone valerate, budesonide, chloroprednisone, chlorprednisone acetate, clescinolone, clobetasol, clobetasol propionate, clobetasol valerate, clobetasone, clobetasone butyrate, clocortelone, cortisone, cortodoxone, craposone butyrate, desonide, desoxymethasone, dexamethasone, desoxycorticosterone acetate, dichlorisone, diflorasone diacetate, diflucortolone valerate, diflurosone diacetate, diflurprednate, fluadrenolone, flucetonide, flucloronide, fluclorolone acetonide, flucortine butylesters, fludroxycortide, fludrocortisone, flumethasone, flumethasone pivalate, flumethasone pivalate, flunisolide, fluocinolone, fluocinolone acetonide, fluocinonide, fluocortin butyl, fluocortolone, fluorometholone, fluosinolone acetonide, fluperolone, fluprednidene acetate, fluprednisolone hydrocortamate, fluradrenolone, fluradrenolone acetonide, flurandrenolone, fluticasone, halcinonide, halobetasol, hydrocortisone, hydrocortisone acetate, hydrocortisone butyrate, hydrocortisone cyclopentylpropionate, hydrocortisone valerate, hydroxyltriamcinolone, medrysone, meprednisone, α-methyl dexamethasone, methylprednisolone, methylprednisolone acetate, mometasone furoate, paramethasone, prednisolone, prednisone, pregnenolone, spironolactone, triamcinolone, triamcinolone acetonide and derivatives, esters, salts and mixtures thereof.

In an embodiment of the present invention, the additional active pharmaceutical ingredient is a hair growth regulator. Suitable hair growth regulators include but are not limited to N-acetylgalactosamine, N-acetylglucosamine, N-acetylmannosamine, acitretin, aminexil, ascomycin, asiatic acid, azelaic acid, benzalkonium chloride, benzethonium chloride, benzydamine, benzyl nicotinate, benzoyl peroxide, benzyl peroxide, betulinic acid, betulonic acid, calcium pantothenate, celastrol, cepharanthine, chlorpheniramine maleate, clinacycin hydrochloride, crataegolic acid, cromakalin, cyproterone acetate, diazoxide, diphenhydramine hydrochloride, dutasteride, estradiol, ethyl-2-hydroxypropanoate, finasteride, D-fucono-1,5-lactone, furoate, L-galactono-1,4-lactone, D-galactosamine, D-glucaro-1,4-lactone, D-glucosamine-3-sulphate, hinokitiol, hydrocortisone, 2-hydroxypropionic acid, isotretinoin, itraconazole, ketoconazole, latanoprost, 2-methyl propan-2-ol, minocyclin, minoxidil, mipirocin, mometasone, oleanolic acid, panthenol, 1,10-phenanthroline, phenyloin, prednisolone, progesterone, propan-2-ol, pseudoterins, resorcinol, selenium sulfide, tazarotene, triclocarbon, triclosan, triiodothyronine, ursolic acid, zinc pyrithione and derivatives, esters, salts and mixtures thereof.

In an embodiment of the present invention, the additional active pharmaceutical ingredient is a hormone. Suitable hormones include but are not limited to methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5a-dihydrotestosterone, testolactone, 17a-methyl-19-nortestosterone, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, progesterone, chlormadinone acetate, megestrol acetate, norgestimate, norgestrel, desogrestrel, trimegestone, gestodene, nomegestrol acetate, progesterone, 5a-pregnan-3b,20a-diol sulfate, 5a-pregnan-3b,20b-diol sulfate, 5a-pregnan-3b.-ol-20-one, 16,5a-pregnen-3b-ol-20-one, 4-pregnen-20b-ol-3-one-20-sulfate, acetoxypregnenolone, anagestone acetate, cyproterone, dihydrogesterone, flurogestone acetate, gestadene, hydroxyprogesterone acetate, hydroxymethylprogesterone, hydroxymethyl progesterone acetate, 3-ketodesogestrel, megestrol, melengestrol acetate, norethisterone and derivatives, esters, salts and mixtures thereof.

In an embodiment of the present invention, the additional active pharmaceutical ingredient is a hydroxy acid. Suitable hydroxy acids include but are not limited to agaricic acid, aleuritic acid, allaric acid, altraric acid, arabiraric acid, ascorbic acid, atrolactic acid, benzilic acid, citramalic acid, citric acid, dihydroxytartaric acid, erythraric acid, galactaric acid, galacturonic acid, glucaric acid, glucuronic acid, glyceric acid, glycolic acid, gularic acid, gulonic acid, hydroxypyruvic acid, idaric acid, isocitric acid, lactic acid, lyxaric acid, malic acid, mandelic acid, mannaric acid, methyllactic acid, mucic acid, phenyllactic acid, pyruvic acid, quinic acid, ribaric acid, ribonic acid, saccharic acid, talaric acid, tartaric acid, tartronic acid, threaric acid, tropic acid, uronic acids, xylaric acid and derivatives, esters, salts and mixtures thereof.

In an embodiment of the present invention, the additional active pharmaceutical ingredient is a keratolytic agent. Suitable keratolytic agents include but are not limited to N-acetylcysteine, azelaic acid, glycolic acid, pyruvic acid, resorcinol, sulfur, salicyclic acid, retinoic acids and derivatives, esters, salts and mixtures thereof.

In an embodiment of the present invention, the additional active pharmaceutical ingredient is a lactam. Suitable lactams include but are not limited to L-galactono-1,4-lactam, L-arabino-1,5-lactam, D-fucono-1,5-lactam, D-glucaro-1,4-lactam, D-glucurono-6,3-lactam, 2,5-tri-O-acetyl-D-glucurono-6,3-lactam, 2-acetamido-2-deoxyglucono-1,5-lactam, 2-acetamido-2-deoxygalactono-1,5-lactam, D-glucaro-1,4:6,3-dilactam, L-idaro-1,5-lactam, 2,3,5,tri-O-acetyl-D-glucaro-1,4-lactam, 2,5-di-O-acetyl-D-glucaro-1,4:6,3-dilactam, D-glucaro-1,5-lactam methyl ester, 2-propionoamide-2-deoxyglucaro-1,5-lactam and derivatives, esters, salts and mixtures thereof.

In an embodiment of the present invention, the additional active pharmaceutical ingredient is a non-steroidal anti-inflammatory agent. Suitable non-steroidal anti-inflammatory agent include but are not limited to azelaic acid, oxicams, piroxicam, isoxicam, tenoxicam, sudoxicam, CP-14,304, salicylates, aspirin, disalcid, benorylate, trilisate, safapryn, solprin, diflunisal, fendosal, acetic acid derivatives, diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac, fenamates, mefenamic, meclofenamic, flufenamic, niflumic, tolfenamic acids, propionic acid derivatives, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofen, pyrazoles, phenylbutazone, oxyphenbutazone, feprazone, azapropazone, trimethazone and derivatives, esters, salts and mixtures thereof.

In an embodiment of the present invention, the additional active pharmaceutical ingredient is a pediculicide. Suitable pediculicides include but are not limited to DDT, lindane, malathion, permethrin and derivatives, esters, salts and mixtures thereof.

In an embodiment of the present invention, the additional active pharmaceutical ingredient is a vasodilator. Suitable vasodilators include but are not limited to ethyl nicotinate, capsicum extract and derivatives, esters, salts and mixtures thereof.

In an embodiment of the present invention, the additional active pharmaceutical ingredient is a wart remover. Suitable wart removers include but are not limited to imiquimod, podophyllotoxin and derivatives, esters, salts and mixtures thereof.

In an embodiment of the present invention, a composition of the present invention includes a propellant in addition to a pharmaceutically acceptable foamable carrier and an active pharmaceutical ingredient. Suitable propellants include but are not limited to nitrous oxide, carbon dioxide, chloropentafluoroethane, dichlorodifluoromethane, nitrogen, propane, iso-butane, n-butane, isopentane, n-pentane, dimethyl ether, trichlorofluoromethane and mixtures thereof. Generally, a propellant makes up between about 3% and about 25% of the total weight of the composition.

In an embodiment of the present invention, the pharmaceutically acceptable foamable carrier comprises a surface-active agent and at least one additional foamable carrier component selected from the group consisting of emulsifiers, fatty alcohols, hydrocarbon alcohols and water.

In an embodiment of the present invention, at least one surface-active agent is a selected from the group consisting of anionic, nonionic, amphoteric, cationic and zwitterionic surface-active agents, and mixtures thereof. Suitable surface-active agents include but are not limited to acyl glutamates, acyl taurates, N-alkoyl sarcosinates, alkyl alkoxy sulfates, alkyl amidopropyl betaines, alkyl arylsulfonates, alkyl amine oxides, alkyl betaines, alkyl carbonates, alkyl carboxyglycinates, alkyl ether carboxylates, alkyl ether phosphates, alkyl ether sulfates, alkyl ether sulfonates, alkyl glyceryl ether sulfates, alkyl glycinates, alkyl phosphates, alkyl succinates, alkyl sulfates, alkyl sulphosuccinates, ammonium alkyl sulphates, ammonium lauryl sulphate, ammonium lauryl sulphosuccinate, ammonium sulfonate, aryl sulfonates, cocamidopropyl betaine, cocodimethyl sulphopropyl betaine, cocomethyl tauride, cocomonoethanolamide, cocodiethanolamide, coco dimethyl carboxymethyl betaine, cocomonoisopropanolamide, disodium laureth sulfosuccinate, dodecylbenzenesulfonate, ethoxylated sorbitan palmitate, ethoxylated sorbitan oleate, ethoxylated sorbitan stearate, fatty acid alkanolamides, fatty acid amino polyoxyethylene sulfates, fatty acids, fatty alcohol ethoxylates, fatty taurides, isothienates, lauryl amine oxide, lauryl betaine, lauryl dimethyl carboxymethyl betaine, lauryl ether carboxylate, lauryl ether sulfate, lauryl glucoside, lauryl sarcosinate, lauryl sulfate, lauryl sulfosuccinate, nonoxynol phosphates, nonyl phenol ethoxylates, olefin sulfonates, octoxynol phosphates, polyethylene glycols, polysorbate 60, sarcosinates, sodium alkyl sulphates, sodium benzene sulfonate, sodium cocamphopropionate, sodium cocoyl isethionate, sodium cumene sulfonate, sodium dodecylbenzene sulphonate, sodium lauroyl isethionate, sodium N-lauryl sarcosinate, sodium laureth sulphate, sodium lauryl sulphate, sodium oleyl succinate, sodium xylene sulfonate, sulfated monoglycerides, sulfobetaines, sulfosuccinates, sultaines, taurates, triethanolamine dodecylbenzene sulphonate, triethanolamine lauryl sulphate, triethanolamine monolauryl phosphate, alkyldimethylbenzyl chloride ammonium salts, alkyldimethylbenzyl bromide ammonium salts, alkyltrimethylbenzyl chloride ammonium salts, alkyltrimethylbenzyl bromide ammonium salts, cetyltrimethylammonium chloride, cetyltrimethylammonium bromide, tetradecyltrimethylammonium chloride, tetradecyltrimethylammonium bromide, alkyldimethyl hydroxyethylammonium chloride, alkyldimethyl hydroxyethyl ammonium bromide, dialkyldimethylammonium chloride, dialkyldimethylammonium bromide, alkylpyridinium salts, lauryl pyridinium chloride, cetyl pyridinium chloride, alkylamidoethyltrimethylammonium ether sulfates, amine oxides, alkylmethylaminoxide, alkylaminoethyldimethylaminoxide and derivatives, esters, salts and mixtures thereof. The concentration of surface-active agents in a composition of the present invention is generally between about 0.1% and about 20% of the total weight of the composition.

In an embodiment of the present invention, at least one additional foamable carrier components is an emulsifier Suitable emulsifiers include but are not limited to sorbitan isostearate, sorbitan sesquioleate, sorbitan trioleate, polyglyceryl-3-diisostearate, polyglycerol esters of oleic/isostearic acid, polyglyceryl-6 hexaricinolate, polyglyceryl-4-oleate, polygylceryl-4 oleate/PEG-8 propylene glycol cocoate, oleamide DEA, sodium glyceryl oleate phosphate, hydrogenated vegetable glycerides phosphate, glyceryl monostearate, diethylaminoethyl alkyl amide phosphate, glyceryl, glycol esters of stearic acid, eicosene copolymer, sorbitan oleate and derivatives, esters, salts and mixtures thereof. When present, the concentration of emulsifiers in a composition of the present invention is generally between about 0.01% and about 10% of the total weight of the composition. In an embodiment of the present invention, at least one additional foamable carrier component is a fatty alcohol, especially a fatty alcohol having between 10 and 22 carbon atoms. Suitable fatty alcohols include but are not limited to cetyl alcohol, stearyl alcohol, lauryl alcohol, myristyl alcohol, palmityl alcohol and mixtures thereof. When present, the concentration of fatty alcohols in a composition of the present invention is generally between 0.01% and 20% by weight of the composition.

In an embodiment of the present invention, the composition of the present invention is alcohol free.

In an embodiment of the present invention, at least one additional foamable carrier component is a hydrocarbon alcohol, especially a hydrocarbon alcohol having between 1 and 10 carbon atom, more preferably between 1 and 6 carbon atoms, especially aliphatic hydrocarbon alcohols. Suitable hydrocarbon alcohols include but are not limited to methanol, ethanol, n-propanol, isopropanol, n-butanol, sec-butanol, isobutanol and t-butanol and mixtures thereof. When present, the concentration of hydrocarbon alcohols in a composition of the present invention is generally between about 0.01% and about 90% of the total weight of the composition.

In an embodiment of the present invention, at least one additional foamable carrier component is water. When present, the concentration of water in a composition of the present invention is generally between about 0.5% and about 95% of the total weight of the composition.

In an embodiment of the present invention, a composition of the present invention includes one or more additional components. Such additional components include but are not limited to anti perspirants, anti-static agents, buffering agents, bulking agents, chelating agents, cleansers, colorants, conditioners, deodorants, diluents, dyes, emollients, fragrances, hair conditioners, humectants, occlusive agents, oils, penetration enhancers, pearlescent aids, perfuming agents, permeation enhancers, pH-adjusting agents, preservatives, protectants, skin penetration enhancers, softeners, solubilizers, sunscreens, sun blocking agents, sunless tanning agents, viscosity modifiers and vitamins. Preferred additional components include buffering agents, emollients, humectants, pH-adjusting agents, sunscreens and sun-blocking agents. As is known to one skilled in the art, in some instances a specific additional component may have more than one activity, function or effect.

In an embodiment of the present invention, the additional component is an anti-static agent, such as a water-insoluble cationic surface-active agent. Suitable anti-static agents include but are not limited to tricetyl methyl ammonium chloride, derivatives thereof and mixtures thereof.

In an embodiment of the present invention, the additional component is a buffering agent. Suitable buffering agents include but are not limited to a citrate buffer, an acetic acid/sodium acetate buffer and a phosphoric acid/sodium phosphate buffer.

In an embodiment of the present invention, the additional component is a conditioner, such as a cationic surface-active agent. Suitable cationic surface-active agents include but are not limited to quaternary ammonium hydroxides, tetramethylammonium hydroxide, alkyltrimethylammonium hydroxides, octyltrimethylammonium hydroxide, dodecyltrimethyl ammonium hydroxide, hexadecyltrimethylammonium hydroxide, cetyltrimethylammonium hydroxide, octyldimethylbenzylammonium hydroxide, decyldimethyl-benzylamrnmonium hydroxide, stearyldimethylbenzylammonium hydroxide, didodecyl dimethyl ammonium hydroxide, dioctadecyldimethylammonium hydroxide, tallow trimethylammonium hydroxide, cocotrimethylammonium hydroxide, cetylpyridinium hydroxide, polyalkylaryl siloxanes, polyalkyl siloxanes, polydimethyl siloxanes, polydiethyl siloxanes, polydimethyl siloxane polymers, polydimethyl siloxane/diphenyl/methylvinylsiloxane copolymers, polydimethylsiloxane/methylvinylsiloxane copolymers and derivatives and mixtures thereof.

In an embodiment of the present invention, the additional component is an emollient. Suitable emollients include but are not limited to mineral oil, lanolin oil, coconut oil, cocoa butter, olive oil, aloe vera extract, jojoba oil, castor oil, fatty acids, fatty alcohols, diisopropyl adipate, isononyl iso-nonanoate, silicone oils, polyethers, C12 to C15 alkyl benzoates, stearic fatty acid, cetyl alcohols, hexadecyl alcohol, dimethyl polysiloxane, polyoxypropylene cetyl ether, polyoxypropylene, and derivatives, esters, salts and mixtures thereof.

In an embodiment of the present invention, the additional component is a fragrance. Suitable fragrances include but are not limited to menthol, eugenol, benzyl salicylate, phenylethyl salicylate, thymol, isoamyl salicylate, phenylethyl salicylate, methyl salicylate, caproic acid, carbaryl and derivatives, esters, salts and mixtures thereof.

In an embodiment of the present invention, the additional component is a humectant. Suitable humectants include but are not limited to, urea, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, polyhydroxy alcohol, sorbitol, glycerol, hexanetriol, propylene glycol, butylene glycol, hexylene glycol, a hexylene glycol derivative, polyethylene glycol, a sugar, a starch, a sugar derivative, a starch derivative, alkoxylated glucose, hyaluronic acid, acetamide monoethanolamine and derivatives, esters, salts and mixtures thereof.

In an embodiment of the acetamide monoethanolamine, present invention, the additional component is a pH-adjusting agent. Suitable pH-adjusting agents include but are not limited to adipic acid, calcium hydroxide, citric acid, glycine, hydrochloric acid, lactic acid, magnesium aluminometasilicates, phosphoric acid, sodium carbonate, sodium citrate, sodium hydroxide, sorbic acid, succinic acid, tartaric acid, and derivatives, salts and mixtures thereof.

In an embodiment of the present invention, the additional component is a preservative. Suitable preservatives include but are not limited to C12 to C15 alkyl benzoates, alkyl p-hydroxybenzoates, aloe vera extract, ascorbic acid, benzalkonium chloride, benzoic acid, benzoic acid esters of C9 to C15 alcohols, butylated hydroxytoluene, diazolidinyl urea, DMDM hydantoin, ethanol, hydroxybenzoate esters, iodopropynyl butylcarbamatemethylparaben, polyoxypropylene butyl ether, polyoxypropylene cetyl ether, potassium sorbate, sodium propionate, sodium benzoate, sodium bisulfite, sorbic acid, and mixtures thereof.

In an embodiment of the present invention, the additional component is a skin penetration enhancer. Suitable skin penetration enhancers include but are not limited to, acyl lactylates, acyl peptides, acylsarcosinates, alkanolamine salts of fatty acids, alkyl benzene sulphonates, alkyl ether sulphates, alkyl sulphates, anionic surface-active agents, benzyl benzoate, benzyl salicylate, butan-1,4-diol, butyl benzoate, butyl laurate, butyl myristate, butyl stearate, cationic surface-active agents, citric acid, cocoamidopropylbetaine, decyl methyl sulfoxide, decyl oleate, dibutyl azelate, dibutyl phthalate, dibenzyl sebacate, dibutyl sebacate, dibutyl suberate, dibutyl succinate, dicapryl adipate, didecyl phthalate, diethylene glycol, diethyl sebacate, diethyl-m-toluamide, di(2-hydroxypropyl) ether, diisopropyl adipate, diisopropyl sebacate, N,N-dimethyl acetamide, dimethyl azelate, N,N-dimethyl formamide, 1,5-dimethyl-2-pyrrolidone, dimethyl sebacate, dimethyl sulphoxide, dioctyl adipate, dioctyl azelate, dioctyl sebacate, 1,4 dioxane, 1-dodecylazacyloheptan-2-one, dodecyl dimethyl amine oxides, ethyl caprate, ethyl caproate, ethyl caprylate, 2-ethyl-hexyl pelargonate, ethyl-2-hydroxypropanoate, ethyl laurate, ethyl myristate, 1-ethyl-2-pyrrolidone, ethyl salicylate, hexyl laurate, 2-hydroxyoctanoic acid, 2-hydroxypropanoic acid, 2-hydroxypropionic acid, isethionates, isopropyl isostearate, isopropyl palmitate, guar hydroxypropyltrimonium chloride, hexan-2,5-diol, khellin, lamepons, lauryl alcohol, maypons, metal salts of fatty acids, methyl nicotinate, 2-methyl propan-2-ol, 1-methyl-2-pyrrolidone, 5-methyl-2-pyrrolidone, methyl taurides, miranol, nonionic surface-active agents, octyl alcohol, octylphenoxy polyethoxyethanol, oleic ethanolamide, pleyl alcohol, pentan-2,4-diol, phenoxyethanol, phosphatidyl choline, phosphine oxides, polyalkoxylated ether glycollates, poly(diallylpiperidinium chloride), poly(dipropyldiailylammonium chloride), polyglycefol esters, polyoxyethylene lauryl ether, polyoxy:polyoxyethylene stearate, polyoxypropylene 15 stearyl ether, poly(vinyl pyridinium chloride), propan-1-ol, propan-2-ol, propylene glycol dipelargonate, pyroglutamic acids, 2-pyrrolidone, pyruvic acids, Quatemium 5, Quaternium 18, Quaternium 19, Quaternium 23, Quaternium 31, Quaternium 40, Quaternium 57, quartenary amine salts, quaternised poly (dimethylaminoethylmethacrylate), quatemised poly (vinyl alcohol), sapamin hydrochloride, sodium cocaminopropionate, sodium dioctyl sulphonsuccinate, sodium laurate, sodium lauryl ether sulphate, sodium lauryl sulphate, sugar esters, sulphosuccinate, tetrahydrofuran, tetrahydrofurfural alcohol, transcutol, oleic acid triethanolamine dodecyl benzene sulphonate, triethanolamine oleate, urea, water esters, salts and mixtures thereof.

In an embodiment of the present invention, the additional component is a solubilizer. Suitable solubilizers include but are not limited to propylene glycol, 1,3-propylene diol, polyethylene glycol, ethanol, propanol, glycerine, dimethyl sulphoxide, dimethyl acetamide, dimethyl formamide, hexylene glycol, water propylene carbonate and derivatives, salts and mixtures thereof.

In an embodiment of the present invention, the additional component is a sunscreen. Suitable sunscreens include but are not limited octyl methoxycinnamate, octyl salicylate, homosalate, octocrylene and derivatives, esters, salts and mixtures thereof.

In an embodiment of the present invention, the additional component is a viscosity modifier. Suitable viscosity modifiers include but are not limited to carbomer, polyethylene glycol, polypropylene glycol, urea, cellulose derivates and mixtures thereof.

In an embodiment of the present invention, a composition of the present invention is packaged in a packaging material and identified in print, in or on the packaging material, that the composition is for use for a need selected from the group consisting of curing a condition, treating a condition, preventing a condition, treating symptoms of a condition, curing symptoms of a condition, ameliorating symptoms of a condition, treating effects of a condition, ameliorating effects of a condition, and preventing results of a condition. In an embodiment of the present invention, the condition is selected from the group consisting of a medical condition and a cosmeceutical condition, especially a skin and/or scalp disease or disorder. Preferably, the skin and/or scalp disease or disorder is selected from the group consisting of rosacea, acne lesions, gangrene, ulcers, decubitis ulcers and tumors.

According to the teachings of the present invention there is also provided a process for preparing a foamable pharmaceutical or cosmeceutical composition of the present invention, comprising: obtaining a mixture of an active pharmaceutical ingredient with a pharmaceutically acceptable foamable carrier; placing the mixture in a pressure-resistant vessel; placing an amount of at least one propellant into the pressure-resistant vessel; and sealing the pressure-resistant vessel, wherein the active pharmaceutical ingredient is a 1-substituted 2-methyl-5-nitro-imidazol, derivatives thereof and mixtures thereof and wherein the pH of the mixture is greater than about 4.5, preferably greater than about 5.0. Since the composition is primarily intended for topical application to the skin or scalp, in a preferred embodiment, the pH of the composition is between about 5.4 and 5.6.

In an embodiment of the process of the present invention, obtaining the mixture includes adjusting the pH of the mixture to be greater than about 4.5, greater than about 5.0, or to be between about 5.4 and 5.6.

In a preferred embodiment of the process of the present invention, the 1-substituted 2-methyl-5-nitro-imidazol active pharmaceutical ingredient is selected from the group consisting of Metronidazole, Tinidazole, Secnidazole, Ornidazole, Benznidazole, derivatives thereof and mixtures thereof. A most preferred active pharmaceutical ingredient is Metronidazole, derivatives of Metronidazole and mixtures thereof.

In an embodiment of the process of the present invention, the pharmaceutically acceptable foamable carrier comprises at least one surface-active agent and at least one additional foamable carrier component selected from the group consisting of emulsifiers, fatty alcohols, hydrocarbon alcohols and water.

In an embodiment of the present invention, suitable surface-active agents include anionic, nonionic, amphoteric, cationic and zwitterionic surface-active agents, and mixtures thereof. Suitable surface-active agents include acyl glutamates, acyl taurates, N-alkoyl sarcosinates, alkyl alkoxy sulfates, alkyl amidopropyl betaines, alkyl arylsulfonates, alkyl amine oxides, alkyl betaines, alkyl carbonates, alkyl carboxyglycinates, alkyl ether carboxylates, alkyl ether phosphates, alkyl ether sulfates, alkyl ether sulfonates, alkyl glyceryl ether sulfates, alkyl glycinates, alkyl phosphates, alkyl succinates, alkyl sulfates, alkyl sulphosuccinates, ammonium alkyl sulphates, ammonium lauryl sulphate, ammonium lauryl sulphosuccinate, ammonium sulfonate, aryl sulfonates, cocamidopropyl betaine, cocodimethyl sulphopropyl betaine, cocomethyl tauride, cocomonoethanolamide, cocodiethanolamide, coco dimethyl carboxymethyl betaine, cocomonoisopropanolamide, disodium laureth sulfosuccinate, dodecylbenzenesulfonate, ethoxylated sorbitan palmitate, ethoxylated sorbitan oleate, ethoxylated sorbitan stearate, fatty acid alkanolamides, fatty acid amino polyoxyethylene sulfates, fatty acids, fatty alcohol ethoxylates, fatty taurides, isothienates, lauryl amine oxide, lauryl betaine, lauryl dimethyl carboxymethyl betaine, lauryl ether carboxylate, lauryl ether sulfate, lauryl glucoside, lauryl sarcosinate, lauryl sulfate, lauryl sulfosuccinate, nonoxynol phosphates, nonyl phenol ethoxylates, olefin sulfonates, octoxynol phosphates, polyethylene glycols, polysorbate 60, sarcosinates, sodium alkyl sulphates, sodium benzene sulfonate, sodium cocamphopropionate, sodium cocoyl isethionate, sodium cumene sulfonate, sodium dodecylbenzene sulphonate, sodium lauroyl isethionate, sodium N-lauryl sarcosinate, sodium laureth sulphate, sodium lauryl sulphate, sodium oleyl succinate, sodium xylene sulfonate, sulfated monoglycerides, sulfobetaines, sulfosuccinates, sultaines, taurates, triethanolamine dodecylbenzene sulphonate, triethanolamine lauryl sulphate, triethanolamine monolauryl phosphate, alkyldimethylbenzyl chloride ammonium salts, alkyldimethylbenzyl bromide ammonium salts, alkyltrimethylbenzyl chloride ammonium salts, alkyltrimethylbenzyl bromide ammonium salts, cetyltrimethylammonium chloride, cetyltrimethylammonium bromide, tetradecyltrimethylammonium chloride, tetradecyltrimethylammonium bromide, alkyldimethylhydroxyethylammonium chloride, alkyldimethyl hydroxyethyl ammonium bromide, dialkyldimethylammonium chloride, dialkyldimethylammonium bromide, alkylpyridinium salts, lauryl pyridinium chloride, cetyl pyridinium chloride, alkylamidoethyltrimethylammonium ether sulfates, amine oxides, alkylmethylaminoxide, alkylaminoethyldimethylaminoxide and derivatives, esters, salts and mixtures thereof.

In an embodiment of the process of the present invention, at least one additional foamable carrier component is an emulsifier. Suitable emulsifiers include but are not limited to sorbitan isostearate, sorbitan sesquioleate, sorbitan trioleate, polyglyceryl-3-diisostearate, polyglycerol esters of oleic/isostearic acid, polyglyceryl-6 hexaricinolate, polyglyceryl-4-oleate, polygylceryl-4 oleate/PEG-8 propylene glycol cocoate, oleamide DEA, sodium glyceryl oleate phosphate, hydrogenated vegetable glycerides phosphate, glyceryl monostearate, diethylaminoethyl alkyl amide phosphate, glyceryl, glycol esters of stearic acid, eicosene copolymer, sorbitan oleate and derivatives, esters, salts and mixtures thereof.

In an embodiment of the process of the present invention, at least one additional foamable carrier component is a fatty alcohol, especially a fatty alcohol having between 10 and 22 carbon atoms. Suitable fatty alcohols include but are not limited to cetyl alcohol, stearyl alcohol, lauryl alcohol, myristyl alcohol, palmityl alcohol and mixtures thereof.

In an embodiment of the process of the present invention, at least one additional foamable carrier component is an aliphatic hydrocarbon alcohol, especially an aliphatic hydrocarbon alcohol having between 1 and 10 carbon atoms (preferably between 1 and 6 carbon atoms. Suitable hydrocarbon alcohols include but are not limited to methanol, ethanol, n-propanol, isopropanol, n-butanol, sec-butanol, isobutanol and t-butanol and mixtures thereof.

In an embodiment of the process of the present invention, at least one additional active pharmaceutical ingredient is combined with the mixture. Suitable additional active pharmaceutical ingredients include but are not limited to active herbal extracts, acaricides, age spot and keratose removing agents, analgesics, local anesthetics, antiacne agents, antiaging agents, antibacterials, antibiotics, antiburn agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antioxidants, antipruritics, antipsoriatic agents, antirosacea agents, antiseborrheic agents, antiseptic, antiswelling, chemotherapeutic agents, corticosteroids, fungicides, hair growth regulators, hormones, hydroxyacids, insecticides, keratolytic agents, lactams, mitocides, non-steroidal anti-inflammatory agents, pediculicides, progestins, sanatives, scabicides, vasodilators and wart removers. As is known to one skilled in the art, in some instances a specific active pharmaceutical ingredient may have more than one activity, function or effect.

In an embodiment of the process of the present invention, at least one additional component is combined with the mixture. Suitable additional components include but are not limited to anti perspirants, anti-static agents, buffering agents, bulking agents, chelating agents, cleansers, colorants, conditioners, deodorants, diluents, dyes, emollients, fragrances, glycerin, hair conditioners, humectants, occlusive agents, oils, penetration enhancers, pearlescent aids, perfuming agents, permeation enhancers, pH-adjusting agents, preservatives, protectants, skin penetration enhancers, softeners, solubilizers, sunscreens, sun blocking agents, sunless tanning agents, viscosity modifiers and vitamins. Especially suitable additional components include buffering agents, emollients, humectants, pH-adjusting agents, sunscreens and sun-blocking agents. As is known to one skilled in the art, in some instances a specific additional component may have more than one activity, function or effect.

According to the teachings of the present invention there is also provided a method of treatment comprising topically administering a therapeutically or cosmeceutically effective amount of an active pharmaceutical ingredient in a foam to an area (e.g. the skin, the scalp, an ulcer, a wound, a tumor or a lesion) of a mammal (human or non-human) in need thereof, the active pharmaceutical ingredient being a 1-substituted 2-methyl-5-nitro-imidazol, derivatives thereof and mixtures thereof, the foam having a pH greater than about 4.5, preferably greater than 5 and more preferably between about 5.4 and 5.6.

In a preferred embodiment of the method of the present invention, the 1-substituted 2-methyl-5-nitro-imidazol active pharmaceutical ingredient is selected from the group consisting of Metronidazole, Tinidazole, Secnidazole, Omidazole, Benznidazole, derivatives thereof and mixtures thereof. A most preferred active pharmaceutical ingredient is Metronidazole, derivatives of Metronidazole and mixtures thereof.

In an embodiment of the process of the present invention, the need is selected from the group consisting of curing a condition, treating a condition, preventing a condition, treating symptoms of a condition, curing symptoms of a condition, ameliorating symptoms of a condition, treating effects of a condition, ameliorating effects of a condition, and preventing results of a condition.

In an embodiment of the method of the present invention, the condition is a medical condition or a cosmeceutical condition, especially a skin and/or scalp disease or disorder, including but not limited to rosacea, acne, lesions, gangrene, ulcers, decubitis ulcers and tumors.

In an embodiment of the method of the present invention, administering is performed by passing a foamable pharmaceutical or cosmeceutical composition containing the at least one active pharmaceutical ingredient from a first volume having a first pressure through a passage into a second volume having a second pressure, the first pressure being greater than the second pressure, so as to effect foaming of the foamable composition. In an embodiment of the present invention, the foamable composition is formulated for topical application to a skin or scalp area and comprises a cosmeceutically or pharmaceutically effective amount of the active pharmaceutical ingredient in a pharmaceutically acceptable foamable carrier. A preferred such foamable composition is a foamable composition of the present invention.

In an embodiment of the method present invention, the concentration of the active pharmaceutical ingredient in the foamable composition used in implementing the method of the present invention ranges between about 0.0001 percent (more preferably 0.001 percent and even more preferably 0.1 percent) and about 2 percent (more preferably 1.5 percent and even more preferably 1 percent) of the total weight of the foamable composition.

In one preferred embodiment of the method of the present invention, the 1-substituted 2-methyl-5-nitro-imidazol is the sole active pharmaceutical ingredient in the foamable composition.

In another preferred embodiment of the method of the present invention, the foamable composition used in implementing the method of the present invention includes at least one additional active pharmaceutical ingredient. Suitable additional active pharmaceutical ingredients include but are not limited to active herbal extracts, acaricides, age spot and keratose removing agents, analgesics, local anesthetics, antiacne agents, antiaging agents, antibacterials, antibiotics, antiburn agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antioxidants, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, fungicides, hair growth regulators, hormones, hydroxyacids, insecticides, keratolytic agents, lactams, mitocides, non-steroidal anti-inflammatory agents, pediculicides, progestins, sanatives, scabicides, vasodilators and wart removers. As is known to one skilled in the art, in some instances a specific active pharmaceutical ingredient may have more than one activity, function or effect.

In an embodiment of the method of the present invention, a foamable composition used in implementing the method of the present invention includes a propellant in addition to a pharmaceutically acceptable foamable carrier and an active pharmaceutical ingredient. Suitable propellants include but are not limited to nitrous oxide, carbon dioxide, nitrogen, hydrocarbons, hydrochlorocarbons, hydrofluorocarbons and mixtures thereof. Generally, the propellant makes up between about 3% and about 25% of the total weight of the foamable composition.

In an embodiment of the method of the present invention, the pharmaceutically acceptable foamable carrier comprises at least one surface-active agent and at least one additional foamable carrier component selected from the group consisting of emulsifiers, fatty alcohols, hydrocarbon alcohols and water.

In an embodiment of the method of the present invention, at least one surface-active agent is a selected from the group consisting of anionic, nonionic, amphoteric, cationic and zwitterionic surface-active agents, and mixtures thereof. Suitable surface-active agents include but are not limited to acyl glutamates, acyl taurates, N-alkoyl sarcosinates, alkyl alkoxy sulfates, alkyl amidopropyl betaines, alkyl arylsulfonates, alkyl amine oxides, alkyl betaines, alkyl carbonates, alkyl carboxyglycinates, alkyl ether carboxylates, alkyl ether phosphates, alkyl ether sulfates, alkyl ether sulfonates, alkyl glyceryl ether sulfates, alkyl glycinates, alkyl phosphates, alkyl succinates, alkyl sulfates, alkyl sulphosuccinates, ammonium alkyl sulphates, ammonium lauryl sulphate, ammonium lauryl sulphosuccinate, ammonium sulfonate, aryl sulfonates, cocamidopropyl betaine, cocodimethyl sulphopropyl betaine, cocomethyl tauride, cocomonoethanolamide, cocodiethanolamide, coco dimethyl carboxymethyl betaine, cocomonoisopropanolamide, disodium laureth sulfosuccinate, dodecylbenzenesulfonate, ethoxylated sorbitan palmitate, ethoxylated sorbitan oleate, ethoxylated sorbitan stearate, fatty acid alkanolamides, fatty acid amino polyoxyethylene sulfates, fatty acids, fatty alcohol ethoxylates, fatty taurides, isothienates, lauryl amine oxide, lauryl betaine, lauryl dimethyl carboxymethyl betaine, lauryl ether carboxylate, lauryl ether sulfate, lauryl glucoside, lauryl sarcosinate, lauryl sulfate, lauryl sulfosuccinate, nonoxynol phosphates, nonyl phenol ethoxylates, olefin sulfonates, octoxynol phosphates, polyethylene glycols, polysorbate 60, sarcosinates, sodium alkyl sulphates, sodium benzene sulfonate, sodium cocamphopropionate, sodium cocoyl isethionate, sodium cumene sulfonate, sodium dodecylbenzene sulphonate, sodium lauroyl isethionate, sodium N-lauryl sarcosinate, sodium laureth sulphate, sodium lauryl sulphate, sodium oleyl succinate, sodium xylene sulfonate, sulfated monoglycerides, sulfobetaines, sulfosuccinates, sultaines, taurates, triethanolamine dodecylbenzene sulphonate, triethanolamine lauryl sulphate, triethanolamine monolauryl phosphate, alkyldimethylbenzyl chloride ammonium salts, alkyldimethylbenzyl bromide ammonium salts, alkyltrimethylbenzyl chloride ammonium salts, alkyltrimethylbenzyl bromide ammonium salts, cetyltrimethylammonium chloride, cetyltrimethylammonium bromide, tetradecyltrimethylammonium chloride, tetradecyltrimethylammonium bromide, alkyldimethyl hydroxyethylammonium chloride, alkyldimethyl hydroxyethyl ammonium bromide, dialkyldimethylammonium chloride, dialkyldimethylammonium bromide, alkylpyridinium salts, lauryl pyridinium chloride, cetyl pyridinium chloride, alkylamidoethyltrimethylammonium ether sulfates, amine oxides, alkylmethylaminoxide, alkylaminoethyldimethylaminoxide and derivatives, esters, salts and mixtures thereof. The concentration of surface-active agents in a foamable composition used in implementing the method of the present invention is generally between about 0.1% and about 20% of the total weight of the foamable composition.

In an embodiment of the method of the present invention, at least one additional foamable carrier components is an emulsifier. Suitable emulsifiers include but are not limited to sorbitan isostearate, sorbitan sesquioleate, sorbitan trioleate, polyglyceryl-3-diisostearate, polyglycerol esters of oleic/isostearic acid, polyglyceryl-6 hexaricinolate, polyglyceryl-4-oleate, polygylceryl-4 oleate/PEG-8 propylene glycol cocoate, oleamide DEA, sodium glyceryl oleate phosphate, hydrogenated vegetable glycerides phosphate, glyceryl monostearate, diethylaminoethyl alkyl amide phosphate, glyceryl, glycol esters of stearic acid, eicosene copolymer, sorbitan oleate and derivatives, esters, salts and mixtures thereof. When present, the concentration of emulsifiers in a foamable composition used in implementing the method of the present invention is generally between about 0.01% and about 10% of the total weight of the foamable composition.

In an embodiment of the method of the present invention, at least one additional foamable carrier components is a fatty alcohol, especially a fatty alcohol having between 10 and 22 carbon atoms. Suitable fatty alcohols include but are not limited to cetyl alcohol, stearyl alcohol, lauryl alcohol, myristyl alcohol, palmityl alcohol and mixtures thereof. When present, the concentration of fatty alcohols in a foamable composition used in implementing the teachings of the present invention is generally between 0.01% and 20% by weight of the foamable composition, more preferably between 0.01% to 10% by weight of the foamable composition.

In an embodiment of the method of the present invention at least one additional foamable carrier component is a hydrocarbon alcohol, especially a hydrocarbon alcohol having between 1 and 10 carbon atoms, more preferably having between 1 and 6 carbon atoms, especially aliphatic hydrocarbon alcohols. Suitable hydrocarbon alcohols include but are not limited to methanol, ethanol, n-propanol, isopropanol, n-butanol, sec-butanol, isobutanol and t-butanol and mixtures thereof. When present, the concentration of hydrocarbon alcohols in a foamable composition used in implementing the method of the present invention is generally between about 0.01% and about 90% of the total weight of the foamable composition.

In an embodiment of the method of the present invention, at least one additional foamable carrier component is water. When present, the concentration of water in a foamable composition used in implementing the method of the present invention is generally between about 0.5% and about 95% of the total weight of the foamable composition.

In an embodiment of the method of the present invention, a foamable composition used in implementing the method of the present invention includes at least one additional component. Suitable additional components include but are not limited to anti perspirants, anti-static agents, buffering agents, bulking agents, chelating agents, cleansers, colorants, conditioners, deodorants, diluents, dyes, emollients, fragrances, glycerin, hair conditioners, humectants, occlusive agents, oils, penetration enhancers, pearlescent aids, perfuming agents, permeation enhancers, pH-adjusting agents, preservatives, protectants, skin penetration enhancers, softeners, solubilizers, sunscreens, sun blocking agents, sunless tanning agents, viscosity modifiers and vitamins. Preferred additional components added to the composition include buffering agents, emollients, humectants, pH-adjusting agents, sunscreens and sun-blocking agents. As is known to one skilled in the art, in some instances a specific additional component may have more than one activity, function or effect.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents and other references mentioned herein are incorporated by reference in their entirety. In case of conflict, the patent specification, including definitions, will control. In addition, the materials, methods, and examples are illustrative only and not intended to be limiting.

### DESCRIPTION OF THE PREFERRED EMBODIMENTS

The present invention is of a foamable pharmaceutical or cosmeceutical composition containing a 1-substituted 2-methyl-5-nitro-imidazole and especially Metronidazole as an active pharmaceutical ingredient in a pharmaceutically acceptable foamable carrier that is useful for the topical delivery of the active pharmaceutical ingredient to a mammal, whether a human or non-human mammal. Embodiments of the composition of the present invention include those applicable to skin as a foam, and to the scalp, as a foam or a mousse. The present invention also includes a process for the preparation of the composition of the present invention. The present invention also includes methods of treatment, substantially using a foamable composition containing a nitroimidazole, such as the composition of the present invention, especially for the treatment of afflictions such as rosacea, acne and skin lesions such as lesions associated with gangrene, ulcers, decubitis ulcers and tumors. As discussed hereinabove, a foam delivery form has many advantages for the topical dispensation of active pharmaceutical ingredients including providing accurate dosage and convenient application. Most importantly, due to the self-cushioning properties of foams, foam compositions allow safe, non-irritating and non-painful topical application to sensitive or damaged areas.

The principles, uses and implementations of the present invention are better understood with reference to the accompanying descriptions and examples.

Before explaining at least one embodiment of the invention in detail, it is to be understood that the invention is not limited in its application to the details set forth herein. The invention can be implemented with other embodiments and can be practiced or carried out in various ways. It is also understood that the phraseology and terminology employed herein is for descriptive purpose and should not be regarded as limiting.

As used herein, the term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “topical active pharmaceutical ingredient” refers to a pharmaceutical or cosmeceutical agent including any natural or synthetic chemical substance, intended for topical application on a surface of a mammal, especially to the skin, and that subsequent to the topical application has, at the very least, at least one desired pharmaceutical effect.

The composition of the present invention is a foamable pharmaceutical or cosmeceutical composition having a pharmaceutically effective amount of an active pharmaceutical ingredient in a pharmaceutically acceptable foamable carrier, the active pharmaceutical ingredient being a 1-substituted 2-methyl-5-nitro-imidazol, a derivative thereof or mixtures thereof, the composition having a pH greater than about 4.5 and preferably greater than about 5.0.

Since the composition of the present invention is primarily intended for topical application to the skin or scalp, in a preferred embodiment, the pH of the composition is between about 5.4 and 5.6.

The pharmaceutically acceptable foamable carrier is formulated to generate foam suitable for topical application to the skin of a patient or is formulated to generate a mousse suitable for topical application to the scalp of a patient.

The active pharmaceutical ingredient in a composition of the present invention is a 1-substituted 2-methyl-5-nitro-imidazole (including all natural and/or synthetic analogues, as well as geometric isomers and stereoisomers of these compounds) as an active pharmaceutical ingredient. Preferred 1-substituted 2-methyl-5-nitro-imidazol active pharmaceutical ingredients include Metronidazole, Tinidazole, Secnidazole, Omidazole, Benznidazole, derivatives thereof and mixtures thereof. Most preferred is Metronidazole, derivatives of Metronidazole and mixtures thereof.

The exact amount of a given active pharmaceutical ingredient in a pharmaceutical or cosmeceutical composition of the present invention is dependent on the condition for which the composition is intended to treat, the exact mode of use and the active pharmaceutical ingredient itself. That said, generally the concentration of the active pharmaceutical ingredient ranges between about 0.0001 percent (more preferably 0.001 percent and even more preferably 0.1 percent) and about 2 percent (more preferably 1.5 percent and even more preferably 1 percent) of the total weight of the composition.

As used herein throughout, the phrase “weight percentage(s)” or “percent” describes the weight percentage(s) of an ingredient of the total weight of a composition containing the ingredient. As used herein the term “about” refers to ±10

In a preferred embodiment of the present invention, the 1-substituted 2-methyl-5-nitro-imidazol is the sole active pharmaceutical ingredient in the composition.

It is known that oftentimes two or more active pharmaceutical ingredients when applied together in one composition act additively, providing an increased effect or more than one desired effect using only one composition. In some instances, two or more active pharmaceutical ingredients when applied together in one composition act synergistically, providing one or more desired effects with exceptional efficacy. Therefore, in another preferred embodiment, the composition of the present invention includes at least one active pharmaceutical ingredient in addition to the 1-substituted 2-methyl-5-nitro-imidazol. Suitable additional active pharmaceutical ingredients include but are not limited to active herbal extracts, acaricides, age spot and keratose removing agents, analgesics, local anesthetics, antiacne agents, antiaging agents, antibacterials, antibiotics, antiburn agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antioxidants, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, fungicides, hair growth regulators, hormones, hydroxyacids, insecticides, keratolytic agents, lactams, mitocides, non-steroidal anti-inflammatory agents, pediculicides, progestins, sanatives, scabicides, vasodilators and wart removers. It is important to note, as is known to one skilled in the art, that in some instances a specific active pharmaceutical ingredient may have more than one activity, function or effect.

Suitable active herbal extracts added as additional active pharmaceutical ingredients to a composition of the present invention include but are not limited to angelica, anise oil, astragali radix, azalea, benzyl acetate, birch tar oil, bomyl acetate, cacumen biotae, camphor, cantharidin, capsicum, cineole, cinnamon bark, cinnamon leaf, citronella, citronellol, citronellyl acetate, citronellyl formate, eucalyptus, eugenyl acetate, flos carthami, fructus mori, garlic, geraniol, geranium, geranyl acetate, habanera, isobutyl angelicate, lavender, ledum latifolium, ledum palustre, lemongrass, limonene, linalool, linalyl acetate, methyl anthranilate, methyl cinnamate, mezereum, neem, nerol, neryl acetate, nettle root extract, oleum ricini, oregano, pinenes, α-pinene, β-pinene, radix angelicae sinesis, radix paenoiae rubra, radix polygoni multiflori, radix rehmanniae, rhizoma pinelliae, rhizoma zingiberis recens, sabadilla, sage, sandalwood oil, saw palmetto extract, semen sesami nigrum, staphysagria, tea tree oil, terpene alcohols, terpene hydrocarbons, terpene esters, terpinene, terpineol, terpinyl acetate and derivatives, esters, salts and mixtures thereof.

Suitable acaricides added as additional active pharmaceutical ingredients to a composition of the present invention include but are not limited to amitraz, flumethrin, fluvalinate and derivatives, esters, salts and mixtures thereof.

Suitable age spot and keratoses removing agent added as additional active pharmaceutical ingredients to a composition of the present invention include but are not limited to hydroxyacids, hydroquinone and derivatives, esters, salts and mixtures thereof.

Suitable analgesics added as additional active pharmaceutical ingredients to a composition of the present invention include but are not limited to benzocaine, butamben picrate, dibucaine, dimethisoquin, dyclonine, lidocaine, pramoxine, tetracaine, salicylates and derivatives, esters, salts and mixtures thereof.

Suitable local anesthetics added as additional active pharmaceutical ingredients to a composition of the present invention include but are not limited to benzocaine, bupivacaine, butamben picrate, chlorprocaine, cocaine, dibucaine, dimethisoquin, dyclonine, etidocaine, hexylcaine, ketamine, lidocaine, mepivacaine, pramoxine, procaine, tetracaine, salicylates and derivatives, esters, salts and mixtures thereof.

In some instances it is useful to provide a composition of the present invention having an antiacne agent in addition to a nitroimidazole such as Metronidazole.

Suitable additional antiacne agents added as additional active pharmaceutical ingredients to a composition of the present invention include but are not limited to N-acetylcysteine, adapalene, azelaic acid, benzoyl peroxide, cholate, clindamycin, deoxycholate, erythromycin, flavinoids, glycolic acid, meclocycline, mupirocin, octopirox, phenoxy ethanol, phenoxy proponol, pyruvic acid, resorcinol, retinoic acid, salicylic acid, scymnol sulfate, sulfacetamide-sulfur, sulfur, tazarotene, tetracycline, tretinoin triclosan and derivatives, esters, salts and mixtures thereof.

Suitable antiaging agents added as additional active pharmaceutical ingredients to a composition of the present invention include but are not limited to melatonin and derivatives, esters, salts and mixtures thereof.

In some instances it is useful to provide a composition of the present invention having an antibiotic in addition to a nitroimidazole such as metronidazole. As is known to one skilled in the art, the term antibiotic includes agents with antimicrobial, antibacterial, antimycotic and/or antiprotozoal activity. Suitable additional antibiotics added as additional active pharmaceutical ingredients to a composition of the present invention to a composition of the present invention include but are not limited to amanfadine hydrochloride, amanfadine sulfate, amikacin, amikacin sulfate, aminoglycosides, amoxicillin, ampicillin, ansamycins, bacitracin, beta-lactams, candicidin, capreomycin, carbenicillin, cephalexin, cephaloridine, cephalothin, cefazolin, cephapirin, cephradine, cephaloglycin, chloramphenicols, chlorhexidine, chlorhexidine gluconate, chlorhexidine hydrochloride, chloroxine, chlorquinaldol, chlortetracycline, chlortetracycline hydrochloride, ciprofloxacin, circulin, clindamycin, clindamycin hydrochloride, clotrimazole, cloxacillin, demeclocycline, diclosxacillin, diiodohydroxyquin, doxycycline, ethambutol, ethambutol hydrochloride, erythromycin, erythromycin estolate, erythromycin stearate, farnesol, floxacillin, gentamicin, gentamicin sulfate, gramicidin, griseofulvin, haloprogin, haloquinol, hexachlorophene, iminocylcline, iodochlorhydroxyquin, kanamycin, kanamycin sulfate, lincomycin, lineomycin, lineomycin hydrochloride, macrolides, meclocycline, methacycline, methacycline hydrochloride, methenamine, methenamine hippurate, methenamine mandelate, methicillin, miconazole, miconazole hydrochloride, minocycline, minocycline hydrochloride, mupirocin, nafeillin, neomycin, neomycin sulfate, netilmicin, netilmicin sulfate, nitrofurazone, norfloxacin, nystatin, octopirox, oleandomycin, orcephalosporins, oxacillin, oxytetracycline, oxytetracycline hydrochloride, parachlorometa xylenol, paromomycin, paromomycin sulfate, penicillins, penicillin G, penicillin V, pentamidine, pentamidine hydrochloride, phenethicillin, polymyxins, quinolones, streptomycin sulfate, tetracycline, tobramycin, tolnaftate, triclosan, trifampin, rifamycin, rolitetracycline, spectinomycin, spiramycin, streptomycin, sulfonamide, tetracyclines, tetracycline, tobramycin, tobramycin sulfate, triclocarbon, triclosan, trimethoprim-sulfamethoxazole, tylosin, vancomycin, yrothricin and derivatives, esters, salts and mixtures thereof.

Suitable antimycotics added as additional active pharmaceutical ingredients to a composition of the present invention include but are not limited to azole compounds, butoconazole, chloroxine, ciclopirox olamine, clotrimazole, econazole, elubiol, fluconazole, griseofulvin, itraconazole, ketoconazole, mafenide acetate, miconazole, nystatin, oxiconazole, sulconazole, terbinafine, terconazole, tioconazole, undecylenic acid and derivatives, esters, salts and mixtures thereof. Suitable antidandruff agents added as additional active pharmaceutical ingredients to a composition of the present invention include but are not limited to aminexil, benzalkonium chloride, benzethonium chloride, 3-bromo-1-chloro-5,5-dimethyl-hydantoin, chloramine B, chloramine T, chlorhexidine, N-chlorosuccinimide, climbazole, 1,3-dibromo-5,5-dimethylhydantoin, 1,3-dichloro-5,5-dimethyl-hydantoin, betulinic acid, betulonic acid, celastrol, crataegolic acid, cromakalin, cyproterone acetate, dutasteride, finesteride, ibuprofen, ketoconozole, oleanolic acid, phenyloin, picrotone olamine, salicylic acid, selenium sulphides, triclosan, triiodothyronine, ursolic acid, zinc gluconate, zinc omadine, zinc pyrithione and derivatives, esters, salts and mixtures thereof.

Suitable antidepressants added as additional active pharmaceutical ingredients to a composition of the present invention include but are not limited to norepinephrine-reuptake inhibitors, selective-serotonin-reuptake inhibitors, monoamine-oxidase inhibitors, serotonin-and-noradrenaline-reuptake inhibitors, corticotropin-releasing factor antagonists, α-adrenoreceptor antagonists, NK1-receptor antagonists, 5-HT_{1A}-receptor agonist antagonists, amitriptyline, desmethylamitriptyline, clomipramine, doxepin, imipramine, imipramine-oxide, trimipramine, adinazolam, amiltriptylinoxide, amoxapine, desipramine, maprotiline, nortriptyline, protriptyline, amineptine, butriptyline, demexiptiline, dibenzepin, dimetacrine, dothiepin, fluacizine, iprindole, lofepramine, melitracen, metapramine, norclolipramine, noxiptilin, opipramol, perlapine, pizotyline, propizepine, quinupramine, reboxetine, tianeptine, binedaline, m-chloropiperzine, citalopram, duloxetine, etoperidone, femoxetine, fluoxetine, fluvoxamine, indalpine, indeloxazine, milnacipran, nefazodone, oxaflazone, paroxetine, prolintane, ritanserin, sertraline, tandospirone, venlafaxine and zimeldine and derivatives, esters, salts and mixtures thereof.

Suitable antihistamines added as additional active pharmaceutical ingredients to a composition of the present invention include but are not limited to chlorcyclizine, diphenhydramine, mepyramine, methapyrilene, tripelennamine and derivatives, esters, salts and mixtures thereof.

Suitable antipruritics added as additional active pharmaceutical ingredients to a composition of the present invention include but are not limited to menthol, methdilazine, trimeprazine, urea and derivatives, esters, salts and mixtures thereof.

Suitable antipsoriatic agents added as additional active pharmaceutical ingredients to a composition of the present invention include but are not limited to 6-aminonicotinamide, 6-aminonicotinic acid, 2-aminopyrazinamide, anthralin, calcipotriene, 6-carbamoylnicotinamide, 6-chloronicotinamide, 2-carbamoylpyrazinamide, corticosteroids, 6-dimethylaminonicotinamide, dithranol, 6-formylaminonicotinamide, 6-hydroxy nicotinic acid, 6-substituted nicotinamides, 6-substituted nicotinic acid, 2-substituted pyrazinamide, tazarotene, thionicotinamide, trichothecene mycotoxins and derivatives, esters, salts and mixtures thereof.

In some instances it is useful to provide a composition of the present invention having an antirosacea agent in addition to a nitroimidazole such as metronidazole. Suitable additional antirosacea agents added as additional active pharmaceutical ingredients to a composition of the present invention include but are not limited to azelaic acid, sulfacetamide and derivatives, esters, salts and mixtures thereof.

Suitable antiseborrheic agents added as additional active pharmaceutical ingredients to a composition of the present invention include but are not limited to glycolic acid, salicylic acid, selenium sulfide, zinc pyrithione and derivatives, esters, salts and mixtures thereof.

Suitable antiviral agents added as additional active pharmaceutical ingredients to a composition of the present invention include but are not limited to acyclovir and derivatives, esters, salts and mixtures thereof.

Suitable chemotherapeutic agents added as additional active pharmaceutical ingredients to a composition of the present invention include but are not limited to daunorubicin, doxorubicin, idarubicin, amrubicin, pirarubicin, epirubicin, mitoxantrone, etoposide, teniposide, vinblastine, vincristine, mitomycin C, 5-FU, paclitaxel, docetaxel, actinomycin D, colchicine, topotecan, irinotecan, gemcitabine cyclosporin, verapamil, valspodor, probenecid, MK571, GF120918, LY335979, biricodar, terfenadine, quinidine, pervilleine A, XR9576 and derivatives, esters, salts and mixtures thereof.

Suitable corticosteroids added as additional active pharmaceutical ingredients to a composition of the present invention include but are not limited to alclometasone dipropionate, amcinafel, amcinafide, amcinonide, beclomethasone, beclomethasone dipropionate, betamethsone, betamethasone benzoate, betamethasone dexamethasone-phosphate, dipropionate, betamethasone valerate, budesonide, chloroprednisone, chlorprednisone acetate, clescinolone, clobetasol, clobetasol propionate, clobetasol valerate, clobetasone, clobetasone butyrate, clocortelone, cortisone, cortodoxone, craposone butyrate, desonide, desoxymethasone, dexamethasone, desoxycorticosterone acetate, dichlorisone, diflorasone diacetate, diflucortolone valerate, diflurosone diacetate, diflurprednate, fluadrenolone, flucetonide, flucloronide, fluclorolone acetonide, flucortine butylesters, fludroxycortide, fludrocortisone, flumethasone, flumethasone pivalate, flumethasone pivalate, flunisolide, fluocinolone, fluocinolone acetonide, fluocinonide, fluocortin butyl, fluocortolone, fluorometholone, fluosinolone acetonide, fluperolone, fluprednidene acetate, fluprednisolone hydrocortamate, fluradrenolone, fluradrenolone acetonide, flurandrenolone, fluticasone, halcinonide, halobetasol, hydrocortisone, hydrocortisone acetate, hydrocortisone butyrate, hydrocortisone cyclopentylpropionate, hydrocortisone valerate, hydroxyltriamcinolone, medrysone, meprednisone, α-methyl dexamethasone, methylprednisolone, methylprednisolone acetate, mometasone furoate, paramethasone, prednisolone, prednisone, pregnenolone, progesterone, spironolactone, triamcinolone, triamcinolone acetonide and derivatives, esters, salts and mixtures thereof.

Suitable hair growth regulators added as additional active pharmaceutical ingredients to a composition of the present invention include but are not limited to N-acetylgalactosamine, N-acetylglucosamine, N-acetylmannosamine, acitretin, aminexil, ascomycin, asiatic acid, azelaic acid, benzalkonium chloride, benzethonium chloride, benzydamine, benzyl nicotinate, benzoyl peroxide, benzyl peroxide, betulinic acid, betulonic acid, calcium pantothenate, celastrol, cepharanthine, chlorpheniramine maleate, clinacycin hydrochloride, crataegolic acid, cromakalin, cyproterone acetate, diazoxide, diphenhydramine hydrochloride, dutasteride, estradiol, ethyl-2-hydroxypropanoate, finasteride, D-fucono-1,5-lactone, furoate, L-galactono-1,4-lactone, D-galactosamine, D-glucaro-1,4-lactone, D-glucosamine-3-sulphate, hinokitiol, hydrocortisone, 2-hydroxypropionic acid, isotretinoin, itraconazole, ketoconazole, latanoprost, 2-methyl propan-2-ol, minocyclin, minoxidil, mipirocin, mometasone, oleanolic acid, panthenol, 1,10-phenanthroline, phenyloin, prednisolone, progesterone, propan-2-ol, pseudoterins, resorcinol, selenium sulfide, tazarotene, triclocarbon, triclosan, triiodothyronine, ursolic acid, zinc pyrithione and derivatives, esters, salts and mixtures thereof.

Suitable hormones added as additional active pharmaceutical ingredients to a composition of the present invention include but are not limited to methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5a-dihydrotestosterone, testolactone, 17a-methyl-19-nortestosterone, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5a-pregnan-3b,20a-diol sulfate, 5a-pregnan-3b,20b-diol sulfate, 5a-pregnan-3b.-ol-20-one, 16,5a-pregnen-3b-ol-20-one, 4-pregnen-20b-ol-3-one-20-sulfate, acetoxypregnenolone, anagestone acetate, cyproterone, dihydrogesterone, flurogestone acetate, gestadene, hydroxyprogesterone acetate, hydroxymethylprogesterone, hydroxymethyl progesterone acetate, 3-ketodesogestrel, megestrol, melengestrol acetate, norethisterone and derivatives, esters, salts and mixtures thereof.

Suitable hydroxyacids added as additional active pharmaceutical ingredients to a composition of the present invention include but are not limited to agaricic acid, aleuritic acid, allaric acid, altraric acid, arabiraric acid, ascorbic acid, atrolactic acid, benzilic acid, citramalic acid, citric acid, dihydroxytartaric acid, erythraric acid, galactaric acid, galacturonic acid, glucaric acid, glucuronic acid, glyceric acid, glycolic acid, gularic acid, gulonic acid, hydroxypyruvic acid, idaric acid, isocitric acid, lactic acid, lyxaric acid, malic acid, mandelic acid, mannaric acid, methyllactic acid, mucic acid, phenyllactic acid, pyruvic acid, quinic acid, ribaric acid, ribonic acid, saccharic acid, talaric acid, tartaric acid, tartronic acid, threaric acid, tropic acid, uronic acids, xylaric acid and derivatives, esters, salts and mixtures thereof.

Suitable keratolytic agents added as additional active pharmaceutical ingredients to a composition of the present invention include but are not limited to N-acetylcysteine, azelaic acid, glycolic acid, pyruvic acid, resorcinol, sulfur, salicyclic acid, retinoic acids and derivatives, esters, salts and mixtures thereof.

Suitable lactams added as additional active pharmaceutical ingredients to a composition of the present invention include but are not limited to L-galactono-1,4-lactam, L-arabino-1,5-lactam, D-fucono-1,5-lactam, D-glucaro-1,4-lactam, D-glucurono-6,3-lactam, 2,5-tri-O-acetyl-D-glucurono-6,3-lactam, 2-acetamido-2-deoxyglucono-1,5-lactam, 2-acetamido-2-deoxygalactono-1,5-lactam, D-glucaro-1,4:6,3-dilactam, L-idaro-1,5-lactam, 2,3,5,tri-O-acetyl-D-glucaro-1,4-lactam, 2,5-di-O-acetyl-D-glucaro-1,4:6,3-dilactam, D-glucaro-1,5-lactam methyl ester, 2-propionoamide-2-deoxyglucaro-1,5-lactam and derivatives, esters, salts and mixtures thereof.

Suitable non-steroidal anti-inflammatory agent added as additional active pharmaceutical ingredients to a composition of the present invention include but are not limited to azelaic acid, oxicams, piroxicam, isoxicam, tenoxicam, sudoxicam, CP-14,304, salicylates, aspirin, disalcid, benorylate, trilisate, safapryn, solprin, diflunisal, fendosal, acetic acid derivatives, diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac, fenamates, mefenamic, meclofenarnic, flufenamic, niflumic, tolfenamic acids, propionic acid derivatives, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofen, pyrazoles, phenylbutazone, oxyphenbutazone, feprazone, azapropazone, trimethazone and derivatives, esters, salts and mixtures thereof.

Suitable pediculocides added as additional active pharmaceutical ingredients to a composition of the present invention include but are not limited to DDT, lindane, malathion, permethrin and derivatives, esters, salts and mixtures thereof.

Suitable vasodilators added as additional active pharmaceutical ingredients to a composition of the present invention include but are not limited to ethyl nicotinate, capsicum extract and derivatives, esters, salts and mixtures thereof.

Suitable wart removers added as additional active pharmaceutical ingredients to a composition of the present invention include but are not limited to imiquimod, podophyllotoxin and derivatives, esters, salts and mixtures thereof.

As used herein, the phrase “pharmaceutically acceptable carrier” describes a carrier that does not cause significant irritation to an organism and does not abrogate the biological activity and properties of the applied active ingredient.

One skilled in the art is well acquainted with various carriers useful for foam compositions. Preferred foamable composition useful in implementing a pharmaceutical or cosmeceutical composition of the present invention include the foamable compositions taught in U.S. Pat. No. 6,627,585, U.S. Pat. No. 6,589,509, U.S. Pat. No. 6,589,518, U.S. Pat. No. 6,368,575, U.S. Pat. No. 6,395,258, U.S. Pat. No. 6,383,472, U.S. Pat. No. 6,080,392, U.S. Pat. No. 6,045,779, U.S. Pat. No. 5,830,438, U.S. Pat. No. 5,690,921, U.S. Pat. No. 5,681,546, U.S. Pat. No. 5,066,481, U.S. Pat. No. 4,834,968, U.S. Pat. No. 4,900,326, U.S. Pat. No. 4,673,569, and especially the U.S. patent application Ser. No. 10/812,356, by the same assignee and references cited therein. Further preferred formulations of foamable compositions of the present invention are described in the Examples below.

An especially preferred foamable carrier useful in implementing a composition of the present invention includes at least one surface-active agent and at least one additional foamable carrier component selected from the group consisting of emulsifiers, fatty alcohols, hydrocarbon alcohols and water.

As used herein, the phrase “surface-active agent” describes a chemical substance that has a lipophilic group and a hydrophilic group and therefore has the property of modifying the interfacial tension of the liquid in which it is dissolved. This phrase typically includes soaps, detergents, emulsifiers, dispersing agents and wetting agents. Suitable surface-active agents include anionic, nonionic, amphoteric, cationic and zwitterionic surface-active agents, and mixtures thereof. Specific suitable surface-active agents include but are not limited to acyl glutamates, acyl taurates, N-alkoyl sarcosinates, alkyl alkoxy sulfates, alkyl amidopropyl betaines, alkyl arylsulfonates, alkyl amine oxides, alkyl betaines, alkyl carbonates, alkyl carboxyglycinates, alkyl ether carboxylates, alkyl ether phosphates, alkyl ether sulfates, alkyl ether sulfonates, alkyl glyceryl ether sulfates, alkyl glycinates, alkyl phosphates, alkyl succinates, alkyl sulfates, alkyl sulphosuccinates, ammonium alkyl sulphates, ammonium lauryl sulphate, ammonium lauryl sulphosuccinate, ammonium sulfonate, aryl sulfonates, cocamidopropyl betaine, cocodimethyl sulphopropyl betaine, cocomethyl tauride, cocomonoethanolamide, cocodiethanolamide, coco dimethyl carboxymethyl betaine, cocomonoisopropanolamide, disodium laureth sulfosuccinate, dodecylbenzenesulfonate, ethoxylated sorbitan palmitate, ethoxylated sorbitan oleate, ethoxylated sorbitan stearate, fatty acid alkanolamides, fatty acid amino polyoxyethylene sulfates, fatty acids, fatty alcohol ethoxylates, fatty taurides, isothienates, lauryl amine oxide, lauryl betaine, lauryl dimethyl carboxymethyl betaine, lauryl ether carboxylate, lauryl ether sulfate, lauryl glucoside, lauryl sarcosinate, lauryl sulfate, lauryl sulfosuccinate, nonoxynol phosphates, nonyl phenol ethoxylates, olefin sulfonates, octoxynol phosphates, polyethylene glycols, polysorbate 60, sarcosinates, sodium alkyl sulphates, sodium benzene sulfonate, sodium cocamphopropionate, sodium cocoyl isethionate, sodium cumene sulfonate, sodium dodecylbenzene sulphonate, sodium lauroyl isethionate, sodium N-lauryl sarcosinate, sodium laureth sulphate, sodium lauryl sulphate, sodium oleyl succinate, sodium xylene sulfonate, sulfated monoglycerides, sulfobetaines, sulfosuccinates, sultaines, taurates, triethanolamine dodecylbenzene sulphonate, triethanolamine lauryl sulphate, triethanolamine monolauryl phosphate, alkyldimethylbenzyl chloride ammonium salts, alkyldimethylbenzyl bromide ammonium salts, alkyltrimethylbenzyl chloride ammonium salts, alkyltrimethylbenzyl bromide ammonium salts, cetyltrimethylammonium chloride, cetyltrimethylammonium bromide, tetradecyltrimethylammonium chloride, tetradecyltrimethylammonium bromide, alkyldimethyl hydroxyethylammonium chloride, alkyldimethyl hydroxyethyl ammonium bromide, dialkyldimethylammonium chloride, dialkyldimethylammonium bromide, alkylpyridinium salts, lauryl pyridinium chloride, cetyl pyridinium chloride, alkylamidoethyltrimethylammonium ether sulfates, amine oxides, alkylmethylaminoxide, alkylaminoethyldimethylaminoxide and derivatives, esters, salts and mixtures thereof. The concentration of surface-active agents in a composition of the present invention is generally between about 0.1% and about 20% of the total weight of the composition.

Emulsifiers suitable as for use as additional foamable carrier components in a composition of the present invention include but are not limited to sorbitan isostearate, sorbitan sesquioleate, sorbitan trioleate, polyglyceryl-3-diisostearate, polyglycerol esters of oleic/isostearic acid, polyglyceryl-6 hexaricinolate, polyglyceryl-4-oleate, polygylceryl-4 oleate/PEG-8 propylene glycol cocoate, oleamide DEA, sodium glyceryl oleate phosphate, hydrogenated vegetable glycerides phosphate, glyceryl monostearate, diethylaminoethyl alkyl amide phosphate, glyceryl, glycol esters of stearic acid, eicosene copolymer, sorbitan oleate and derivatives, esters, salts and mixtures thereof. When present, the concentration of emulsifiers in a composition of the present invention is generally between about 0.01% and about 10% of the total weight of the composition.

As used herein, the phrase “fatty alcohol” describes a non-aromatic hydrocarbon alcohol having at least ten carbon atoms and no more than one alcohol group. Fatty alcohols suitable as for use as additional foamable carrier components in a composition of the present invention include but are not limited to fatty alcohols having between 10 and 22 carbon atoms. Such fatty alcohols include but are not limited to cetyl alcohol, stearyl alcohol, lauryl alcohol, myristyl alcohol, palmityl alcohol and mixtures thereof. When present, the concentration of fatty alcohols in a composition of the present invention is generally between 0.01% and 20% by weight of the composition.

As used herein, the phrase “hydrocarbon alcohol” describes a hydrocarbon that is substituted by one or more hydroxyl groups. Suitable hydrocarbon alcohols are preferably aliphatic alcohols and preferably have between 1 and 10 carbon atoms and more preferably between 1 and 6 carbon atoms, especially aliphatic hydrocarbon alcohols. The aliphatic chain is branched or un-branched, saturated or unsaturated, preferably saturated. Such hydrocarbon alcohols include but are not limited to methanol, ethanol, n-propanol, isopropanol, n-butanol, sec-butanol, isobutanol and t-butanol and mixtures thereof. When present, the concentration of hydrocarbon alcohols in a composition of the present invention is generally between about 0.01% and about 90% of the total weight of the composition.

When present, the concentration of water in a composition of the present invention is generally between about 0.5% and about 95% of the total weight of the composition.

In some embodiments, in addition to the active pharmaceutical ingredient and the pharmaceutically acceptable foamable carrier, a foamable composition of the present invention also includes a propellant. A propellant is used to dispense the composition from a container and to assist in the foaming of the composition. One-skilled in the art is well acquainted with various propellants and uses thereof with foamable compositions see, for example, the references cited above. It is important to note that in some cases a specific propellant also serves at least one additional function, for example, as a component of the carrier and/or as a preservative.

Propellants suitable for use with a composition of the present invention include but are not limited to nitrous oxide, carbon dioxide, chloropentafluoroethane, dichlorodifluoromethane, nitrogen, propane, iso-butane, n-butane, isopentane, n-pentane, dimethyl ether, trichlorofluoromethane and mixtures thereof. Generally, a propellant makes up between about 3% and about 25% of the total weight of a composition of the present invention.

It is often desired, especially when providing a cosmeceutical composition, to provide a composition with additional useful properties. Therefore, in some embodiments, a composition of the present invention includes, in addition to a foamable carrier and an active pharmaceutical ingredient, at least one additional component. One skilled in the art is well acquainted with the use and combination of various additional components in foamable compositions, see for example the references cited above. It is important to note that in some cases a specific additional component also serves as a component of the carrier or serves two or more additional functions. For example, in a specific composition ethanol can serve as a propellant, a preservative, as a viscosity modifier and as a solubilizer. Typical additional components include but are not limited to anti perspirants, anti-static agents, buffering agents, bulking agents, chelating agents, cleansers, colorants, conditioners, deodorants, diluents, dyes, emollients, fragrances, hair conditioners, humectants, occlusive agents, oils, penetration enhancers, pearlescent aids, perfuming agents, permeation enhancers, pH-adjusting agents, preservatives, protectants, skin penetration enhancers, softeners, solubilizers, sunscreens, sun blocking agents, sunless tanning agents, viscosity modifiers and vitamins. Exceptionally preferred additional components include buffering agents, emollients, humectants and pH-adjusting agents. For compositions useful in treating rosacea, it is exceptionally preferred to add sunscreens and/or sun-blocking agents as additional components. It is important to note, as is known to one skilled in the art, that in some instances a specific additional component may have more than one activity, function or effect.

Suitable anti-static agents added as additional components to a composition of the present invention include but are not limited to water-insoluble cationic surface-active agents such as tricetyl methyl ammonium chloride, derivatives thereof and mixtures thereof.

Suitable buffering agents added as additional components to a composition of the present invention include but are not limited to citrate buffers, acetic acid/sodium acetate buffers and a phosphoric acid/sodium phosphate buffers.

Suitable conditioners added as additional components to a composition of the present invention include but are not limited to cationic surface-active agen, quaternary ammonium hydroxides, tetramethylammonium hydroxide, alkyltrimethylammonium hydroxides, octyltrimethylammonium hydroxide, dodecyltrimethyl ammonium hydroxide, hexadecyltrimethylammonium hydroxide, cetyltrimethylammonium hydroxide, octyldimethylbenzylammonium hydroxide, decyldimethyl-benzylammonium hydroxide, stearyldimethylbenzylammonium hydroxide, didodecyl dimethyl ammonium hydroxide, dioctadecyldimethylammonium hydroxide, tallow trimethylammonium hydroxide, cocotrimethylammonium hydroxide, cetylpyridinium hydroxide, polyalkylaryl siloxanes, polyalkyl siloxanes, polydimethyl siloxanes, polydiethyl siloxanes, polydimethyl siloxane polymers, polydimethyl siloxane/diphenyl/methylvinylsiloxane copolymers, polydimethylsiloxane/methylvinylsiloxane copolymers and derivatives and mixtures thereof.

Suitable emollients added as additional components to a composition of the present invention include but are not limited to mineral oil, lanolin oil, coconut oil, cocoa butter, olive oil, aloe vera extract, jojoba oil, castor oil, fatty acids, fatty alcohols, diisopropyl adipate, isononyl iso-nonanoate, silicone oils, polyethers, C12 to C15 alkyl benzoates, stearic fatty acid, cetyl alcohols, hexadecyl alcohol, dimethyl polysiloxane, polyoxypropylene cetyl ether, polyoxypropylene and derivatives, esters, salts and mixtures thereof.

Suitable fragrances added as additional components to a composition of the present invention include but are not limited to menthol, benzyl alcohol, eugenol, phenoxyethanol, isopropyl palmitate, isopropyl myristate, benzyl salicylate, phenylethyl salicylate, thymol, isoamyl salicylate, phenylethyl salicylate, benzoic acid, benzyl benzoate, methyl salicylate, phenol, oleic acid, caproic acid, carbaryl and derivatives, esters, salts and mixtures thereof.

Suitable humectants added as additional components to a composition of the present invention include but are not limited to guanidine, urea, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, polyhydroxy alcohol, sorbitol, glycerol, hexanetriol, propylene glycol, butylene glycol, hexylene glycol, a hexylene glycol derivative, polyethylene glycol, a sugar, a starch, a sugar derivative, a starch derivative, alkoxylated glucose, hyaluronic acid, lactamide monoethanolamine, acetamide monoethanolamine and derivatives, esters, salts and mixtures thereof.

Suitable pH-adjusting agents added as additional components to a composition of the present invention include but are not limited to adipic acid, calcium hydroxide, citric acid, glycine, hydrochloric acid, lactic acid, magnesium aluminometasilicates, phosphoric acid, sodium carbonate, sodium citrate, sodium hydroxide, sorbic acid, succinic acid, tartaric acid, and derivatives, salts and mixtures thereof.

Suitable preservatives added as additional components to a composition of the present invention include but are not limited to C12 to C15 alkyl benzoates, alkyl p-hydroxybenzoates, aloe vera extract, ascorbic acid, benzalkonium chloride, benzoic acid, benzoic acid esters of C9 to C15 alcohols, butylated hydroxytoluene, castor oil, cetyl alcohols, chlorocresol, citric acid, cocoa butter, coconut oil, diazolidinyl urea, diisopropyl adipate, dimethyl polysiloxane, DMDM hydantoin, ethanol, fatty acids, fatty alcohols, hexadecyl alcohol, hydroxybenzoate esters, iodopropynyl butylcarbamate, isononyl iso-nonanoate, jojoba oil, lanolin oil, methylparaben, mineral oil, oleic acid, olive oil, polyethers, polyoxypropylene butyl ether, polyoxypropylene cetyl ether, potassium sorbate, silicone oils, sodium propionate, sodium benzoate, sodium bisulfite, sorbic acid, stearic fatty acid, vitamin E, vitamin E acetate and derivatives, esters, salts and mixtures thereof.

Suitable skin penetration enhancers added as additional components to a composition of the present invention include but are not limited to acetone, acyl lactylates, acyl peptides, acylsarcosinates, alkanolamine salts of fatty acids, alkyl benzene sulphonates, alkyl ether sulphates, alkyl sulphates, anionic surface-active agents, benzyl benzoate, benzyl salicylate, butan-1,4-diol, butyl benzoate, butyl laurate, butyl myristate, butyl stearate, cationic surface-active agents, citric acid, cocoamidopropylbetaine, decyl methyl sulfoxide, decyl oleate, dibutyl azelate, dibutyl phthalate, dibenzyl sebacate, dibutyl sebacate, dibutyl suberate, dibutyl succinate, dicapryl adipate, didecyl phthalate, diethylene glycol, diethyl sebacate, diethyl-m-toluamide, di(2-hydroxypropyl) ether, diisopropyl adipate, diisopropyl sebacate, N,N-dimethyl acetamide, dimethyl azelate, N,N-dimethyl formamide, 1,5-dimethyl-2-pyrrolidone, dimethyl sebacate, dimethyl sulphoxide, dioctyl adipate, dioctyl azelate, dioctyl sebacate, 1,4 dioxane, 1-dodecylazacyloheptan-2-one, dodecyl dimethyl amine oxides, ethyl caprate, ethyl caproate, ethyl caprylate, 2-ethyl-hexyl pelargonate, ethyl-2-hydroxypropanoate, ethyl laurate, ethyl myristate, 1-ethyl-2-pyrrolidone, ethyl salicylate, hexyl laurate, 2-hydroxyoctanoic acid, 2-hydroxypropanoic acid, 2-hydroxypropionic acid, isethionates, isopropyl isostearate, isopropyl palmitate, guar hydroxypropyltrimonium chloride, hexan-2,5-diol, khellin, lamepons, lauryl alcohol, maypons, metal salts of fatty acids, methyl nicotinate, 2-methyl propan-2-ol, 1-methyl-2-pyrrolidone, 5-methyl-2-pyrrolidone, methyl taurides, miranol, nonionic surface-active agents, octyl alcohol, octylphenoxy polyethoxyethanol, oleic ethanolamide, pleyl alcohol, pentan-2,4-diol, phenoxyethanol, phosphatidyl choline, phosphine oxides, polyalkoxylated ether glycollates, poly(diallylpiperidinium chloride), poly(dipropyldiallylammonium chloride), polyglycerol esters, polyoxyethylene lauryl ether, polyoxy:polyoxyethylene stearate, polyoxypropylene 15 stearyl ether, poly(vinyl pyridinium chloride), propan-1-ol, propan-2-ol, propylene glycol dipelargonate, pyroglutamic acids, 2-pyrrolidone, pyruvic acids, Quatemium 5, Quatemium 18, Quatemium 19, Quatemium 23, Quatemium 31, Quatemium 40, Quaternium 57, quartenary amine salts, quaternised poly (dimethylaminoethylmethacrylate), quaternised poly (vinyl alcohol), sapamin hydrochloride, sodium cocaminopropionate, sodium dioctyl sulphonsuccinate, sodium laurate, sodium lauryl ether sulphate, sodium lauryl sulphate, sugar esters, sulphosuccinate, tetrahydrofuran, tetrahydrofurfural alcohol, transcutol, triethanolamine dodecyl benzene sulphonate, triethanolamine oleate, urea, water and derivatives, esters, salts and mixtures thereof.

Suitable solubilizers added as additional components to a composition of the present invention include but are not limited to propylene glycol, 1,3-propylene diol, polyethylene glycol, ethanol, propanol, glycerine, dimethyl sulphoxide, dimethyl -acetamide, dimethyl formamide, hexylene glycol, propylene carbonate, polysorbate, water-soluble vitamins, e.g., nicotinamide, ascorbic acid or pyridoxine HCl, and their derivatives, salts and mixtures thereof.

Suitable sunscreens added as additional components to a composition of the present invention include but are not limited to benzophenone-3, benzophenone-6, benzophenone-8, benzophenone-12, octyl methoxycinnamate, octyl salicylate, homosalate, methyl anthranilate, octocrylene and derivatives, esters, salts and mixtures thereof.

Suitable viscosity modifiers added as additional components to a composition of the present invention include but are not limited to carbomer, polyethylene glycol, polypropylene glycol, sodium xylene sulphonate, sodium toluene sulphonate, urea and mixtures thereof. The composition of the present invention is formulated to deliver the active pharmaceutical ingredient. It is therefore preferred that a composition of the present invention be packaged in a packaging material and identified in print, in or on the packaging material, for use for a need selected from the group consisting of curing a condition, treating a condition, preventing a condition, treating symptoms of a condition, curing symptoms of a condition, ameliorating symptoms of a condition, treating effects of a condition, ameliorating effects of a condition, and preventing results of a condition. The specific condition and specific use identified is dependent on the exact formulation of a specific composition, especially the nature and amount of the one or more active pharmaceutical ingredients therein.

When one of the active pharmaceutical ingredients is Metronidazole, typical skin and/or scalp disease or disorder identified include but are not limited to rosacea, acne, lesions, gangrene, ulcers, decubitis ulcers and tumors.

In a preferred embodiment, a composition includes both metronidazole and a sunscreen or sun-blocking agents and the skin and/or scalp disease or disorder identified in print is rosacea.

The present invention also provides a method of treatment, the method of treatment substantially being topically administering a therapeutically or cosmeceutically effective amount of an active pharmaceutical ingredient in a foam to an area (e.g. the skin, the scalp, an ulcer, a wound, a tumor or a lesion) of a mammal (human or non-human) in need thereof, the active pharmaceutical ingredient being a 1-substituted 2-methyl-5-nitro-imidazol, derivatives thereof and mixtures thereof, the foam having a pH greater than about 4.5, preferably greater than 5 and more preferably between about 5.4 and 5.6.

Preferably the 1-substituted 2-methyl-5-nitro-imidazol active pharmaceutical ingredient is selected from the group consisting of Metronidazole, Tinidazole, Secnidazole, Omidazole, Benznidazole, derivatives thereof and mixtures thereof. Most preferred is Metronidazole, derivatives of Metronidazole and mixtures thereof.

By “need” is meant a need selected from the group consisting of curing a condition, treating a condition, preventing a condition, treating symptoms of a condition, curing symptoms of a condition, ameliorating symptoms of a condition, treating effects of a condition, ameliorating effects of a condition, and preventing results of a condition. A specific condition and specific use is dependent on the exact formulation of a specific foamable composition, especially the nature and amount of the one or more active pharmaceutical ingredients therein. In a preferred embodiment of the present invention, the condition is a medical condition or a cosmeceutical condition, especially a skin and/or scalp or disorder, including but not limited to rosacea, acne, lesions, gangrene, ulcers, decubitis ulcers and tumors, especially when one of the active pharmaceutical ingredients is metronidazole.

In another preferred embodiment, a composition includes both metronidazole and a sunscreen or sun blocking agent and the skin and/or scalp disease or disorder is rosacea.

A prophylactically, therapeutically, pharmaceutically or cosmeceutically effective amount, as used herein, means an amount of an active pharmaceutical ingredient needed to achieve the desired outcome, which is generally to prevent, alleviate or ameliorate the condition or symptoms of the condition which is being treated. Determination of the effective amount, and consequently the dose and dose frequency, is within the capability of one skilled in the art, especially in light of the detailed disclosure provided herein. Factors in determining the effective amount vary with severity of the condition as well as such factors as the concentration of the active pharmaceutical ingredient or ingredients, the subject being treated, the severity of the condition, the age, body weight and response of an individual patient and the judgment of the prescribing physician.

Generally, administering a composition of the present invention is effected by passing the foamable composition from a first volume having a first pressure (e.g., a pressurized container) through a passage (e.g., a valve) into a second volume having a second pressure, the second pressure being lower than the first pressure (e.g., the outside environment) so as to effect foaming of the foamable composition. Preferably the foamable composition is administered onto a surface. In an embodiment of the present invention, the foamable composition is formulated for topical application to a skin or scalp area and comprises a cosmeceutically or pharmaceutically effective amount of the active pharmaceutical ingredient in a pharmaceutically acceptable foamable carrier. A preferred such foamable composition for implementing the method of treatment of the present invention is a foamable composition of the present invention, as described hereinabove.

In one preferred embodiment of the method of the present invention, the 1-substituted 2-methyl-5-nitro-imidazol is the sole active pharmaceutical ingredient in the foamable composition.

In another preferred embodiment of the method of the present invention, the foamable composition used in implementing the method of the present invention includes at least one additional active pharmaceutical ingredient. Such an additional active pharmaceutical ingredient functions additively or synergistically with the 1-substituted 2-methyl-5-nitro-imidazol active pharmaceutical ingredient so as to provide an added value to the composition, increase efficacy, increase safety, lower toxicity, increase acceptance, increased patient compliance, perform additional pharmaceutical, cosmeceutical, or cosmetic functions, and/or add functionalities.

Suitable additional active pharmaceutical ingredients include but are not limited to active herbal extracts, acaricides, age spot and keratose removing agents, analgesics, local anesthetics, antiacne agents, antiaging agents, antibacterials, antibiotics, antiburn agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antioxidants, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, fungicides, hair growth regulators, hormones, hydroxyacids, insecticides, keratolytic agents, lactams, mitocides, non-steroidal anti-inflammatory agents, pediculicides, progestins, sanatives, scabicides, vasodilators and wart removers. As noted hereinabove, in some instances a specific active pharmaceutical ingredient may have more than one activity, function or effect.

In another preferred embodiment of the method of the present invention, the foamable composition used in implementing the method of the present invention includes at least one additional component. Such components provide an added value to the composition, increase acceptance, increased patient compliance, perform additional pharmaceutical, cosmeceutical, or cosmetic functions, and/or add functionalities. Suitable additional components include but are not limited to anti perspirants, anti-static agents, buffering agents, bulking agents, chelating agents, cleansers, colorants, conditioners, deodorants, diluents, dyes, emollients, fragrances, glycerin, hair conditioners, humectants, occlusive agents, oils, penetration enhancers, pearlescent aids, perfuming agents, permeation enhancers, pH-adjusting agents, preservatives, protectants, skin penetration enhancers, softeners, solubilizers, sunscreens, sun blocking agents, sunless tanning agents, viscosity modifiers and vitamins. Exceptionally preferred additional components include buffering agents, emollients, humectants, pH-adjusting agents, sunscreens and sun-blocking agents. As is known to one skilled in the art, in some instances a specific additional component may have more than one activity, function or desired effect.

A preferred process for the preparation of a foamable composition of the present invention involves obtaining a mixture of an active pharmaceutical ingredient with a pharmaceutically acceptable foamable carrier; placing the mixture in a pressure-resistant vessel; placing an amount of at least one propellant into the pressure-resistant vessel; and sealing the pressure-resistant vessel, wherein the active pharmaceutical ingredient is a 1-substituted 2-methyl-5-nitro-imidazol, derivatives thereof and mixtures thereof and wherein the pH of the mixture is greater than about 4.5, preferably greater than about 5.0. Since the composition is primarily intended for topical application to the skin or scalp, in a preferred embodiment, the pH of the composition is between about 5.4 and 5.6.

In an embodiment of the process of the present invention, obtaining the mixture includes adjusting the pH of the mixture to be greater than about 4.5, greater than about 5.0, or to be between about 5.4 and 5.6. Adjusting pH appropriately is well within the ability of one skilled in the art and generally involves adding components such as buffering agents or pH-adjusting agents to the mixture.

Types and specific examples of suitable active pharmaceutical ingredients, suitable foamable carriers and suitable propellants are listed hereinabove.

In some embodiments of the present invention, one or more additional active pharmaceutical ingredients are added to the mixture. Types and specific examples of suitable additional active pharmaceutical ingredients are listed hereinabove.

In some embodiments of the present invention, one or more additional components are added to the mixture. Types and specific examples of suitable additional components are listed hereinabove.

Generally, but not necessarily, obtaining the mixture includes combining ingredients that are not entirely soluble in water in a non-aqueous solvent and combining water-soluble ingredients in water to obtain two clear solutions, and subsequently mixing the two solutions.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above description illustrate the invention in a non-limiting fashion.

Generally, the nomenclature used herein and the laboratory procedures utilized in the present invention include chemical and analytical techniques with which on skilled in the art is familiar. Unless otherwise defined, technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

Preparation of a Foamable Metronidazole Composition of the Present on for Compositions I-XV:

Metronidazole is combined and mixed with propylene glycol, stearyl alcohol, lauryl sulfate and ethanol to make a waterless solution. The mixture is heated to about 45° C. an aqueous succinate buffer solution added and the mixture stirred until a clear solution is obtained. The solution is poured into an aerosol can. A valve is attached to the can. The hydrocarbon propellant is added to the can and an actuator assembled on the valve.

Using this process, foamable compositions I through XV below are prepared.

Compositions I-V

| | Ingredient | I | II | III | IV | V |
|---|---|---|---|---|---|---|
| 1 | Metronidazole | 0.75 | 0.75 | 1 | 1 | 0.1 |
| 2 | propylene glycol | 2 | 1.5 | 2.5 | 1.5 | 2.5 |
| 3 | stearyl alcohol | 1.6 | 1.6 | 2 | 2 | 1 |
| 4 | lauryl sulfate | 0.4 | 0.4 | 0.4 | 0.4 | 0.4 |
| 7 | propellant | 5 | 5 | 5 | 5 | 5 |
| 5 | ethanol | 59 | 60 | 54 | 55 | 61 |
| 6 | water + succinate buffer | 31 | 31 | 35 | 35 | 30 |
| | (pH 5.5) | | | | | |

Compositions VI-X

| | Ingredient | VI | VII | VIII | IX | X |
|---|---|---|---|---|---|---|
| 1 | Metronidazole | 1 | 0.1 | 0.75 | 0.5 | 0.75 |
| 2 | propylene glycol | 2 | 2 | 2 | 2 | 2 |
| 3 | stearyl alcohol | 2 | 3 | 4 | 2 | 3 |
| 4 | lauryl sulfate | 1 | 1 | 1 | 0.6 | 0.6 |
| 5 | propellant | 7 | 7 | 7 | 7 | 7 |
| 6 | ethanol | 7 | 7 | 7 | 8 | 8 |
| 7 | water + succinate buffer (pH 5.5) | 80 | 80 | 78 | 80 | 79 |

Compositions XI-XV

| | Ingredient | XI | XII | XIII | XIV | XV |
|---|---|---|---|---|---|---|
| 1 | Metronidazole | 0.1 | 0.75 | 0.75 | 1 | 0.75 |
| 2 | Propylene glycol | 2 | 1.5 | 2.5 | 1.5 | 2.5 |
| 3 | Stearyl alcohol | 1.6 | 1.6 | 2 | 2 | 1 |
| 4 | Lauryl sulfate | 0.4 | 0.4 | 0.4 | 0.4 | 0.4 |
| 5 | Propellant | 5 | 5 | 5 | 5 | 5 |
| 6 | Ethanol | 60 | 60 | 54 | 55 | 60 |
| 7 | Water + ? [symbol] | 31 | 31 | 35 | 35 | 30 |
| | succinate buffer | | | | | |
| | (pH 5.5) | | | | | |

Preparation of a Foamable Metronidazole Composition of the Present Invention for Compositions XVI-XVIII

Metronidazole is combined with water, methyl paraben and propyl paraben while heating to about 45° C. to make a solution. The propylene glycol and carboxymethyl cellulose sodium suspension is added and mixed until clear solution is obtained. Then Tween 20, Oleth 20 and glycerine added. The solution is cooled to room temperature. The phosphoric acid is added to adjust pH. The solution is poured into an aerosol can. A valve is attached to the can. The hydrocarbon propellant is added to the can and an actuator is assembled on the valve.

Using this process, foamable compositions XVI through XVIII below are prepared.

Compositions XVI-XVIII

| | Ingredient | XVI | XVI | XVII | XVIII |
|---|---|---|---|---|---|
| 1 | Metronidazole | 0.75 | 0.75 | 1 | 2 |
| 2 | Methyl Paraben | 0.18 | 0.18 | 0.18 | 0.18 |
| 3 | Propyl Paraben | 0.02 | 0.02 | 0.02 | 0.02 |
| 4 | Propylene Glycol | 3.0 | 10 | 10 | 15 |
| 5 | Sodium-CMC | 0.5 | 0.5 | 0.5 | 0.5 |
| 6 | Tween 20 | 5.0 | 5.0 | 5 | 5 |
| 7 | Oleth 20 | 5.0 | 5.0 | 5 | 5 |
| 8 | Phosphoric acid | Qs to | Qs to | Qs to | Qs to |
| | | pH 5.5 | pH 5.5 | pH 5.5 | pH 5.5 |
| 9 | Glycerine | 7 | 0 | 5 | 0 |
| 10 | Water | Qs to | Qs to | Qs to | Qs to |
| | | 100% | 100% | 100% | 100% |
| 11 | Propellant | 5 | 5 | 5 | 5 |

Preparation of a Foamable Metronidazole Composition of the Present Invention for Compositions XVIII-IXX

Metronidazole is mixed and heated to about 75° C. with water, propylene glycol, glycerin, methyl paraben and propyl paraben to make a solution. Stearic acid and Glyceryl monostearate is melted at 75° C. and then added to the aqueous solution. The mixture is mixed and homogenized to make an emulsion and then cooled to a room temperature. An appropriate amount of Triethnolamine is added to adjust pH. Then Tween 20 is added. The cream is poured into an aerosol can. A valve is attached to the can. The hydrocarbon propellant is added to the can and an actuator assembled on the valve.

Using this process, foamable compositions XIII through IXX below are prepared.

Compositions XIII-IXX

| | Ingredient | XIII | XVI | XVII | XVIII |
|---|---|---|---|---|---|
| 1 | Metronidazole | 0.5 | 0.75 | 1 | 2 |
| 2 | Stearic acid | 9 | 9 | 9 | 9 |
| | Propylene glycol | 0 | 0 | 0 | 5 |
| 3 | Glycerin | 2.6 | 2.6 | 2.6 | 0 |
| 4 | Methylparaben | 0.12 | 0.12 | 0.12 | 0.12 |
| 5 | Propylparaben | 0.04 | 0.04 | 0.04 | 0.04 |
| 6 | Glyceryl monostearate | 5 | 5 | 5 | 5 |
| 7 | Triethnolamine | Qs to | Qs to | Qs to | Qs to |
| | | pH 6.5 | pH 6.5 | pH 6.5 | pH 6.5 |
| 8 | Tween 20 | 5 | 5 | 5 | 5 |
| 9 | Water | Qs to | Qs to | Qs to | Qs to |
| | | 100% | 100% | 100% | 100% |
| 11 | Propellant | 5 | 5 | 5 | 5 |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described with reference to specific embodiments thereof, many alternatives, modifications and variations will be apparent to those skilled in the art. Accordingly, it is intended that the present invention embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent and patent application was specifically and individually indicated to be incorporated herein by reference. In addition, citation or identification of any reference in this application shall not be construed as an admission that such reference is available as prior art to the present invention.

What is claimed is:

1. A foamable pharmaceutical or cosmeceutical composition for topical application to a mammalian patient comprising a pharmaceutically effective amount of an active pharmaceutical ingredient in a pharmaceutically acceptable foamable carrier, said active pharmaceutical ingredient being a 1-substituted 2-methyl-5-nitro-imidazol, a derivative thereof or mixtures thereof, the composition having a pH greater than about 4.5, wherein said composition is devoid of urea and/or a derivative thereof and/or hydroxy acid.

2. The composition of claim 1, wherein the composition is alcohol free.

3. The composition of claim 1, wherein said 1-substituted 2-methyl-5-nitro-imidazol is selected from the group consisting of metronidazole, tinidazole, secnidazole, ornidazole, benznidazole, derivatives thereof and mixtures thereof.

4. The composition of claim 1, wherein said active pharmaceutical ingredient is selected from the group consisting of metronidazole, derivatives of metronidazole and mixtures thereof.

5. The composition of claim 1, wherein the concentration of said active pharmaceutical ingredient ranges between about 0.0001 percent and about 2 percent of the total weight of the composition.

6. The composition of claim 1, wherein said active pharmaceutical ingredient is the sole active pharmaceutical ingredient in the composition.

7. The composition of claim 1, further comprising at least one additional active pharmaceutical ingredient.

8. The composition of claim 6, wherein said additional active pharmaceutical ingredient is selected from the group consisting of active herbal extracts, acaricides, age spot and keratose removing agents, analgesics, local anesthetics, antiacne agents, antiaging agents, antibacterials, antibiotics, antiburn agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antioxidants, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, fungicides, hair growth regulators, hormones, hydroxyacids, insecticides, keratolytic agents, lactams, mitocides, non-steroidal anti-inflammatory agents, pediculicides, progestins, sanatives, scabicides, vasodilators and wart removers.

9. The composition of claim 1, further comprising a propellant.

10. The composition of claim 9, wherein said propellant is selected from the group consisting of nitrous oxide, carbon dioxide, chloropentafluoroethane, dichlorodifluoromethane, nitrogen, propane, iso-butane, n-butane, isopentane, n-pentane, dimethyl ether, trichlorofluoromethane and mixtures thereof.

11. The composition of claim 1, wherein said pharmaceutically acceptable foamable carrier comprises at least one surface-active agent and at least one additional foamable carrier component selected from the group consisting of emulsifiers, fatty alcohols, hydrocarbon alcohols and water.

12. The composition of claim 1, further comprising at least one additional component selected from the group consisting of an anti perspirant, anti-static agent, a buffering agent, a bulking agent, a chelating agent, a cleanser, a colorant, a conditioners, a deodorant, a diluent, a dye, an emollient, a fragrance, glycerin, a hair conditioner, a humectant, an occlusive agent, oil, a penetration enhancer, a pearlescent aid, a perfuming agent, a permeation enhancer, a pH-adjusting agent, a preservative, a protectant, a skin penetration enhancer, a softener, a solubilizer, a sunscreen, a sun blocking agent, a sunless tanning agent, a viscosity modifier and a vitamin.

13. The composition of claim 1, for the treatment of a skin and/or scalp disease or disorder.

14. The composition of claim 13, wherein said skin and/or scalp disease or disorder is selected from the group consisting of rosacea, acne, lesions, gangrene, ulcers, decubitis ulcers and tumors.

15. A method of treatment of a skin or scalp disease or disorder comprising topically administering a therapeutically or cosmeceutically effective amount of an active pharmaceutical ingredient in a foamable composition to an area of a mammal in need thereof, said active pharmaceutical ingredient being a 1-substituted 2-methyl-5-nitro-imidazol, derivatives thereof and mixtures thereof, said foam having a pH greater than about 4.5 and wherein said composition is devoid of urea and/or its derivatives and/or hydroxy acid.

16. The method of claim 15, wherein the foamable composition is alcohol free.

17. The method of claim 15, wherein said active pharmaceutical ingredient is selected from the group consisting of metronidazole, tinidazole, secnidazole, omidazole, benznidazole, derivatives thereof and mixtures thereof.

18. The method of claim 15, wherein said active pharmaceutical ingredient is selected from the group consisting of metronidazole, derivatives of metronidazole and mixtures thereof.

19. The method of claim 15, wherein the medical condition is a skin and/or scalp disease or disorder.

20. The method of claim 19, wherein said skin and/or scalp disease or disorder is selected from the group consisting of rosacea, acne, lesions, gangrene, ulcers, decubitis ulcers and tumors.

21. The method of claim 15, wherein the concentration of said active pharmaceutical ingredient ranges between about 0.0001 percent and about 5 percent of the total weight of said foamable composition.

22. The method of claim 15, wherein said active pharmaceutical ingredient is a sole active pharmaceutical ingredient in said foamable composition.

23. The method of claim 15, said foamable composition further comprising at least one additional active pharmaceutical ingredient selected from the group consisting of active herbal extracts, acaricide, age spot and keratoses removing agents, analgesics, local anesthetics, antiacne agents, antiaging agents, antibacterials, antibiotics, antiburn agents, antidandruff agents, antidepressant, antidermatitis agents, antiedemics, antihistamines, antihelminths, antihyperkeratolyte agents, antiinflammatory agents, antiirritants, antilipemics, antimicrobials, antimycotics, antioxidants, antipruritics, agents, antipsoriatic agents, antirosacea agents, antiseborrheic agents, antiseptic, antiswelling agents, antiviral agents, antiyeast agents, astringents, topical cardiovascular agents, chemotherapeutics, corticosteroids, fungicides, hair growth regulators, hormones, hydroxyacids, insecticides, keratolytics, lactams, mitocide, non-steroidal anti-inflammatory agents, pediculicide, progestins, sanatives, scabicide, vasodilators and wart removers.

24. The method of claim 15, said foamable composition further comprising a propellant.

25. The method of claim 15, wherein said pharmaceutically acceptable foamable carrier comprises at least one surface-active agent and at least one additional foamable carrier component selected from the group consisting of emulsifiers, fatty alcohols, hydrocarbon alcohols and water.

26. The method of claim 15, said foamable composition further comprising at least one additional component selected from the group consisting of an anti perspirant, anti-static agent, a buffering agent, a bulking agent, a chelating agent, a cleanser, a colorant, a conditioners, a deodorant, a diluent, a dye, an emollient, a fragrance, glycerin, a hair conditioner, a humectant, an occlusive agent, oil, a penetration enhancer, a pearlescent aid, a perfuming agent, a permeation enhancer, a pH-adjusting agent, a preservative, a protectant, a skin penetration enhancer, a softener, a solubilizer, a sunscreen, a sun blocking agent, a sunless tanning agent, a viscosity modifier and a vitamin.
